(19) United States
(12) Patent Application Publication
(10) Pub. No.: US 2019/0172587 A1
(43) Pub. Date: Jun. 6, 2019

(54) APPARATUS AND METHOD FOR PREDICTING DISEASE RISK OF METABOLIC DISEASE

(71) Applicant: SEOUL NATIONAL UNIVERSITY R&DB FOUNDATION, Seoul (KR)

(72) Inventors: Sue Kyung PARK, Seoul (KR); Jong Hyo KIM, Seoul (KR); Joo Ho TAI, Seoul (KR); Choong Hyun AHN, Seoul (KR); Seo Kyung AN, Goyang-si (KR); Jeoung Bin CHOI, Seoul (KR)

(21) Appl. No.: 16/236,947

(22) Filed: Dec. 31, 2018

Related U.S. Application Data
(63) Continuation-in-part of application No. PCT/KR2017/015773, filed on Dec. 29, 2017.

(30) Foreign Application Priority Data
Dec. 30, 2016 (KR) 10-2016-0183851

(51) Int. Cl. G16H 50/30; G16H 50/50; G16H 50/70; G06N 7/00; G06F 17/18

(52) U.S. Cl. CPC G16H 50/30; G16H 50/50; G06F 17/18; G06N 7/005; G16H 50/70

(57) ABSTRACT

Provided is an apparatus for predicting a disease risk of a metabolic disorder. The apparatus includes: a machine learning model generating unit which generates a machine learning model which learns a degree of a relationship between at least one of a plurality of state variables and genetic information and a disease risk of metabolic disorders with the plurality of state variables including a living condition variable and a health condition variable of a patient with a metabolic disorder, generic information, and the disease risk of the metabolic disorders as inputs; an information input unit which receives a subject state variable and subject genetic information of the subject; and a disease risk predicting unit which predicts a subject disease risk of the subject by applying the subject state variable and the subject genetic information of the subject to the machine learning model.

### TECHNICAL FIELD

The present application relates to an apparatus and is method for predicting a disease risk of metabolic disorders (hypertension, diabetes, obesity, and metabolic syndrome).

### BACKGROUND ART

A representative example of diseases for which a health risk prediction tool is implemented and intervention Tor a high risk group is actively performed is breast cancer. The breast cancer risk evaluation model implemented in the West may be roughly divided into three categories.

One of them is a model fur predicting an absolute incidence possibility by a joint risk of a baseline risk and a risk factor in a general population, the other is a method for predicting an incidence possibility according to a relative risk size of the risk factor, and the third is a model specified for prediction of hereditary breast cancer to predict the BRCA gene mutation possibility based on a family history or a breast cancer possibility based on the BRCA gene mutation possibility.

Currently, in Korea, the Korea academy of family medicine develops a Korean style health risk prediction tool and a personalized health management program service is provided in the website <Health iN> of National Health insurance Service for the people who have received a health checkup through National Health Insurance Service by applying the health risk prediction tool.

However the health risk prediction toot provided by National Health Insurance Service has proven its specificity (validity) for morality, but lacks an analysis for the causes of individual deaths. Further, since a main purpose of this tool is to find and implement correctable health risk factors, there is a limit that is inappropriate to measure the current health condition of the individuals.

Therefore, a method for predicting a future disease possibility based on individual's lifestyle and health condition is required.

A related art of the present disclosure is disclosed in Korean Unexamined Patent Application Publication No. 10-2004-0012368 (published on Feb. 11, 2004).

### DISCLOSURE

### Technical Problem

The present disclosure has been made in an effort to solve the problems of the related art and provide an apparatus and a method for predicting a disease risk of a metabolic disorder which may construct on algorithm to predict an incidence risk of obesity, diabetes, or hypertension which is a current disease state related to the metabolic disorders using individual's lifestyle, health condition, and generic information and predict the end-of-life condition such as chronic heart disease risk related to the chronic disease or death based on the constructed algorithm.

The present disclosure has been made in an effort to solve the problems of the related art and provide an apparatus and a method for predicting a disease risk of a metabolic disorder which may construct an artificial neural network based prediction model and a disease risk prediction model based on a statistical probability model, based on a generic data source and follow-up data source of Ansan-Anseong Cohort which is a part of Korean Genome and Epidemiology Study performed by Korea Centers for Disease Control and Prevention and follow-up data sources and predict the prevalence risk of disease associated with the current metabolic syndrome using the constructed model and predict a probability of a future risk of developing metabolic disorders such as hypertension, diabetes, obesity, and metabolic syndrome to indicate a guideline for guiding a lifestyle change for primary prevention.

The present disclosure has been made in an effort to solve the problems of the related art and provide an apparatus and a method for predicting a disease risk of a metabolic disorder which construct a disease prediction model based on an artificial neural network and a disease occurrence prediction model based on a statistical probability, calculate a probability value of a subject for each disease occurrence risk, and construct a personalized preventive management service model through a visualization algorithm.

However, objects to be achieved by various exemplary embodiments of the present invention are not limited to the technical objects as described above and other technical objects may be present.

### Technical Solution

According to an aspect of the present disclosure, an apparatus for predicting a disease risk of metabolic disorder includes: a machine learning model generating unit which generates a machine learning model which learns a degree of a relationship between at least one of a plurality of state variables and genetic information and a disease risk of metabolic disorders with the plurality of state variables including a living condition variable and a health condition variable of a patient with a metabolic disorder, generic information, and the disease risk of the metabolic disorders as inputs; an information input unit which receives a subject state variable and subject genetic information of the subject; and a disease risk predicting unit which predicts a subject disease risk of the subject by applying the subject state variable and the subject genetic information of the subject to the machine learning model.

According to an exemplary embodiment of the present disclosure, the apparatus for predicting a disease risk of metabolic disorder may further include: a statistical probability model generating unit which generates a statistical probability model probabilistically representing the disease risk of the metabolic disorders depending on whether there are at least one of the plurality of state variables and genetic information or a value, with the plurality of state variables, the genetic information, and the disease risk of the metabolic disorder of a pattern with the metabolic disorder as inputs; and a disease risk predicting unit which predicts a subject disease risk of the subject by applying the subject state variables and the subject genetic information to the machine learning, model and the statistical probability model.

According to an exemplary embodiment of the present disclosure, the statistical probability model generating unit may include a basic statistical probability model generating which has the plurality of state variables, the genetic information and a disease risk of the metabolic disorders of the patient with the metabolic disorder as inputs, selects at least one state variable associated with the metabolic disorder among the plurality of state variables, and generates a basic statistical probability model probabilistically representing the disease risk of the metabolic disorder for whether there is at least one state variable or the value; and a weight statistical probability model generating unit which applies a weight to the disease risk of the metabolic disorder depending on whether there is genetic information associated with the metabolic disorder to generate a statistical probability model from the basic statistical probability model.

According to an exemplary embodiment of the present disclosure, the machine learning model may perform first learning to learn a degree of a relationship between an input layer and a hidden layer when a first state variable among the plurality of state variables is assumed to be the input layer and a second state variable among the plurality of state variables is assumed to be the hidden layer and perform second learning a degree of a relationship between the hidden layer and an output layer when the hidden layer and the genetic in formation are assumed to be the input layer and the disease risk is assumed to be the output layer to learn a degree of a relationship between at least one of the plurality of state variables and genetic information and the disease risk of the metabolic disorder.

According to an exemplary embodiment of the present disclosure, the machine learning model may perform first learning to learn a degree of a relationship between an input layer and a hidden layer when a previous state variable of the plurality of state variables is assumed to be the input layer and a current state variable of the plurality of state variables is assumed to be the hidden layer and perform second learning a degree of a relationship between the hidden layer and an output layer when the hidden layer and the genetic information are assumed to be the input layer and the disease risk is assumed to be the output layer to learn a degree of a relationship between at least one of the plurality of state variables and genetic information and the disease risk of the metabolic disorder.

According to an exemplary embodiment of the present disclosure, the machine learning model may perform first learning to leant a degree of a relationship between an input layer and a hidden layer when a first state variable among the plurality of state variables and a previous hidden layer are assumed to be the input layer and a second state variable or a current state variable among the plurality of state variables is assumed to be the hidden layer and perform second learning a degree of a relationship between the hidden layer and an output layer when the hidden layer and the genetic information are assumed to be the input layer and the disease risk is assumed to be the output layer to learn a degree of a relationship between at least one of the plurality of suite variables and genetic information and the disease risk of the metabolic disorder and the first learing learns the degree of the relationship between the input layer and the bidden layer based on Equation 1.

hₜ=tan h(Wₕₕhₜ₋₁+Wₓₕ·xₜ) [Equation 1]

In this case, hₜ is a hidden layer at a timing t, hₜ₋₁ is a hidden layer at a previous timing, xₜ is a first state variable, Wₕₕ is a first weight representing a degree of a first type of relationship between the input laser and the hidden layer, and Wₓₕ is a second weight representing a degree of a second type of relationship between the input layer and the hidden layer.

According to an exemplary embodiment of the present disclosure, the second learning may learn a degree of a relationship between the hidden layer and the output layer, based on Equations 1 and 2.

y=sigmoid(W_{yh}hₜ+W_{yz}z) [Equation 2]

In this case, y is the output layer, W_{yh} is a third weight representing a degree of the relationship between the hidden layer and the output layer, hₜ is a hidden layer, W_{yz} is a fourth weight representing a degree of the relationship between the genetic information of the input layer and the output layer, and z is the genetic information of the input layer.

According to an exemplary embodiment of the present disclosure, the machine leaning model generating unit may update the weight to an error generated when the machine learning model to learn a degree of the relationship between at least one of the plurality of state variables and genetic information and the disease risk of the metabolic disorders is generated, based on Equation 3.

E=(t−y)²+λ∥W∥₂² [Equation 3]

E is a detected error of the machine learning model generating unit, t is whether the metabolic disorder occurs, y is a disease risk predicted through a machine learning model, and ∥W∥₂² is an L2 regular expression for preventing overfitting due to the error.

According to an exemplary embodiment of the present disclosure, the disease risk predicting unit may visualize a disease risk prediction result of the subject based on a predetermined classification category.

According to an exemplary embodiment of the present disclosure, it is possible to provide disease preventive management information associated wish a disease risk prediction result of the subject.

According to an exemplary embodiment of the present disclosure, when the metabolic disorder is hypertension, the statistical probability model generating unit may generate a statistical probability model probabilistically representing a disease risk of the hypertension according to values of the plurality of state variables including at least five of age, an education level, a monthly average income, anemia, proteinuria, glucose in urine, cholesterol, an amount of sodium intake, an amount of potassium intake, a drinking status, a smoking status, hyperlipidemia, fatty liver, allergies, arthritis, an uric acid level in blood, a family history of metabolic disorder, and whether to exercise.

According to an exemplary embodiment of the present disclosure, when the metabolic disorder is obesity, the statistical probability model generating unit may generate a statistical probability model probabilistically representing a disease risk of the obesity according to values of the plurality of state variables including at least five of age, an education level, a past history of hyperlipidemia, a past history of myocardial infarction, a past history of fatty liver, a past history of cholecystitis, a past history of allergy, a thyroid gland disease, arthritis, a blood pressure whether to exercise, an amount of sodium intake compared with an amount of energy intake, an amount of protein intake, an amount of fat intake, proteinuria, a total cholesterol a fasting blood sugar, a drinking status, a smoking status, an uric acid level in blood, and a family history of metabolic disorder.

According to an exemplars embodiment of the present disclosure, when the metabolic disorder is diabetes, the statistical probability model generating unit may generate a statistical probability model probabilistically representing a disease risk of the diabetes according to values of the plurality of state variables including at least five of an education level, a marriage state, an occupation, an income, a gender an age, a past history of hypertension, a past history of hyperlipidemia, a past history of myocardial infarction, a past history of chronic gastritis, a past history of fatty liver, a past history of cholecystitis, a past history of chronic bronchitis, a past history of asthma, a past history of allergy, arthritis, a past history of osteoporosis, a past history of cataract, a past history of depressive disorder, a past history of thyroid gland disease, a number of exposure to passive smoking, total alcohol intakes, a number of exercises, an age of first birth, a past history of gestational diabetes, a past history of induced abortion, a past history of birth of fetal macrosomia, whether to take oral contraceptive pill, a family history of diabetes, a family history of angina pectoris, a family history of stroke, a current subjective health condition, a quality of sleep hematuria, fat, carbohydrate, vitamin, zinc, a weight, a waist size, a hip circumference, a pulse rate, a diastolic blood pressure, a systolic blood pressure, and a body mass index.

According to an exemplary embodiment of the present disclosure, when the metabolic disorder is a metabolic syndrome, the statistical probability model generating unit may generate a statistical probability model probabilistically representing a disease risk of metabolic syndrome according to values of the plurality of state variables including at least five of an age, a gender, an education level, a monthly average income, ALT, anemia, proteinuria, sodium intake, potassium intake, energy intake, whether to exercise, a pack year of smoking, a past history of myocardial infarction, a past history of fatty liver, a past history of cholecystitis, an allergic disease, a past history of thyroid gland disease, arthritis, an uric acid level in blood, and a family history of metabolic disorder.

According to an exemplary embodiment of the present disclosure, a method for predicting a disease risk of metabolic disorder includes: generating a machine learning model which learns a degree of a relationship between at least one of a plurality of state variables and genetic information and a disease risk of metabolic disorders with the plurality of state variables including a living condition variable and a health condition variable of a patient with a metabolic disorder, generic information, and the disease risk of the metabolic disorders as inputs, receiving a subject state variable and subject genetic information of the subject; and predicting a subject disease risk of the subject by applying the subject state variable and the subject genetic information of the subject to the machine learning model.

The above-described solving means are merely illustrative but should not be construed as limiting the present disclosure. In addition to the above-described exemplary embodiments, additional exemplary embodiments may be further provided in the drawings and the detailed description of the present disclosure.

### Advantageous Effects

According to the above-described solution of the present disclosure, it is possible to confirm a current possible disease probability of a metabolic disorder such as hypertension, diabetes, obesity, and metabolic syndrome, based on individual's state variables and genetic information confirm which group of four risk groups (low, normal, high, very high) current states of subjects who are not yet diagnosed with the disease belong, and predict a future incidence probability of hypertension, diabetes, obesity, or metabolic syndrome based on that to prevent and treat the disease through early diagnosis.

According to the above-described solution of the present disclosure, it is possible to construct an artificial neural network based prediction model and a disease risk prediction model based on a statistical probability model based on a generic data source and follow-up data source of Ansan-Anseong Cohort which is a part of Korean Genome and Epidemiology Study performed by Korea Centers for Disease Control and Prevention and predict the prevalence risk of disease associated with the current metabolic syndrome using the constructed model and predict a future incidence risk probability of metabolic disorders such as hypertension, diabetes, obesity, and metabolic syndrome to indicate a guideline for guiding a lifestyle change for primary prevention.

According to the above-described solution of the present disclosure, it is possible to construct a disease prediction model based on an artificial neural network and a disease prediction model based on a statistical probability, calculate a probability value of a subject for each disease incidence risk, and construct a personalized preventive management service model through a visualization algorithm.

According to the above-described solution of the present disclosure, since subjects having hypertension, diabetes, metabolic syndrome have a higher risk of accompanying other metabolic disorders in the future, a treatment possibility is increased through early diagnosis and the incidence of complications, a cardiovascular disease, a chronic heart disease, or a death risk due to the metabolic disorder are further reduced so that the quality of life of individual may be improved.

According to the above-described solution of the present disclosure, it is possible to apply to health care field of general population in the community or be utilized to select high risk groups in clinical and be utilized tor products utilizing WEB and APP of the risk prediction model.

### DESCRIPTION OF DRAWINGS

FIG. 1 is a schematic system of an apparatus for predicting a disease of metabolic disorders according to an exemplary embodiment of the present disclosure.

FIG. 2 is a schematic diagram of an apparatus for predicting a disease of metabolic disorders according to an exemplary embodiment of the present disclosure.

FIGS. 3A to 3G are views for explaining an example that a disease of metabolic disorders is predicted based on a statistical probability model generating unit according to an exemplary embodiment of the present disclosure.

FIG. 4 is a view schematically illustrating a process of predicting a subject disease risk of a subject by applying a subject state variable of the subject and subject genetic information to a machine learning model and a statistical probability model according to an exemplary embodiment of the present disclosure.

FIG. 5 is an exemplary view for explaining an example of evaluating a risk through a disease prevalence risk incidence risk probability prediction and death risk of a statistical probability model generating unit according to an exemplary embodiment of the present disclosure.

FIG. 6 is a view for explaining an example of a process of predicting a metabolic disorder risk according to an exemplary embodiment of the present disclosure.

FIG. 7 is a view illustrating clustering a plurality of metabolic disorders according to an exemplary embodiment of the present disclosure.

FIG. 8 is a view visualizing a guideline map for a risk of metabolic disorders according to an exemplary embodiment of the present disclosure.

FIGS. 9A to 9P are exemplary views for explaining a statistical probability model of disease risk prediction of individual metabolic disorders according to an exemplary embodiment of the present disclosure.

FIG. 10 is a schematic flowchart of a method for predicting a disease risk of metabolic disorders according to an exemplary embodiment of the present disclosure.

### BEST MODE

Hereinafter, the present disclosure will be described snore fully hereinafter with reference to the accompanying drawings, in which exemplary embodiments of the present disclosure are shown so that those skilled in the art can easily carry out the present invention. However, the present disclosure can be realized in various different forms, and is not limited to the exemplary embodiments described herein. Accordingly, in order to clearly explain the present disclosure in the drawings, portions not related to the description are omitted. Like reference numerals designate like elements throughout the specification.

Throughout this specification and the claims that follow, when it is described that an element is “coupled” to another element the element may be “directly coupled” to the other element or “electrically coupled” to the other element through a third element.

Through the specification of the present disclosure, when one member is located “on”, “above”, “on an upper portion”, “below”, “under”, and “on a lower portion” of the other member, the member may be adjacent to the other member or a third member may be disposed between the above two members.

In the specification, unless explicitly described to the contrary, the word “comprise” and variations such as “comprises” or “comprising” will be understood to imply the inclusion of stated elements but not she exclusion of any other elements.

The present disclosure relates to a metabolic disorder risk predicting apparatus which predicts a disease risk of a subject based on an artificial neural network based disease prediction model and a statistical probability based disease prediction model.

According to an exemplary embodiment of the present disclosure, FIG. 1 is a schematic system diagram of an apparatus of predicting a disease of metabolic disorders according to an exemplary embodiment of the present disclosure. Referring to FIG. 1, the apparatus 100 for predicting a disease of metabolic disorders may interwork with a disease prediction server 200 through a network, but is not limned thereto. For example, the disease prediction server 200 may include a generic data source of Ansan-Anseong Cohort which is a part of Korea Genome and Epidemiology Study performed by Korea Centers for Disease Control and Prevention and follow-up data obtained through first to seventh tracking. The disease prediction server 200 may provide information of a generic data source of Ansan-Anseong Cohort which is a part of Korean Genome and Epidemiology Study performed by Korea Centers for Disease Control and Prevention and follow-up data to the apparatus 100 for predicting a disease of metabolic disorders through a network.

According to an exemplary embodiment, the apparatus 100 for predicting a disease of metabolic disorders is a device including at least one interface device for example, may be all kinds of wireless communication devices such as a smart phone, a smart pad, a tablet PC, a wearable device, a personal communication system (PCS), a global system for mobile communication (GSM), a personal digital cellular (PDC), a personal handyphone system (PHS), a personal digital assistant (PDA), an international mobile communication (IMT)-2000, code division multiple access (CDMA)-2000, W-code division multiple access (W-CDMA), a wireless broadband internet (Wibro) terminal and fixed terminals such as a desktop computer and a smart TV. For example, a disease prediction application of metabolic disorders which provide prediction information of a disease risk to a user may be installed and driven in the device, but is not limited thereto.

A method for predicting a disease of metabolic disorders described below may be performed in the apparatus 100 for predicting a disease of metabolic disorders. As another example, each step of the method for predicting a disease of metabolic disorders may be performed in the disease prediction server 200. As still another example, some of the steps of the method for predicting a disease of metabolic disorders may be performed in the apparatus 100 for predicting a disease of metabolic disorders and the remaining steps may be performed in the disease prediction server 200. For example, the apparatus 100 for predicting a disease of metabolic disorders perform only functions of receiving a user input, transmitting the received user input to a server, and displaying information transmitted from the server on a screen in response to the user input as some steps and the remaining steps of the method for predicting a disease of metabolic disorders may be performed in the disease prediction server 200. Hereinafter, for the convenience of description, an example that the method for predicting a disease of metabolic disorders is performed in the apparatus 100 for predicting a disease of metabolic disorders will be described.

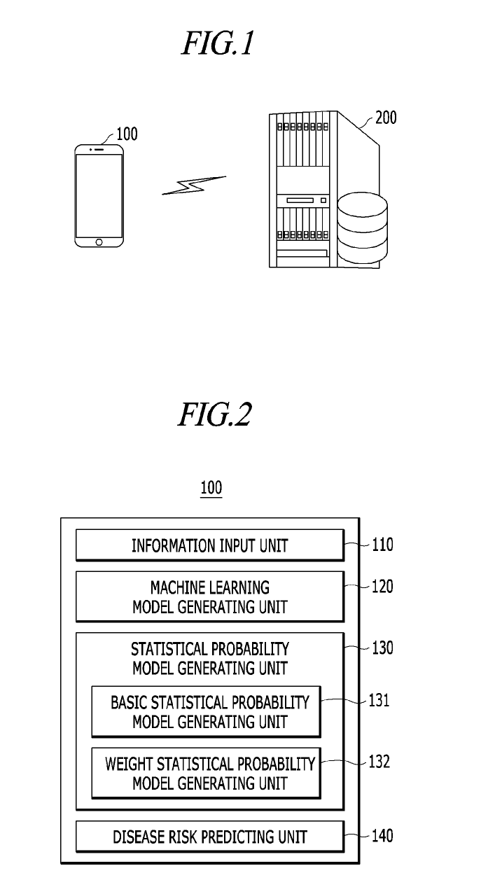

FIG. 2 is a schematic diagram of an apparatus for predicting a disease of metabolic disorders according to an exemplary embodiment of the present disclosure. Referring to FIG. 2, the apparatus 100 for predicting a disease of metabolic disorder may include an information input unit 110, a machine learning model generating unit 120, a statistical probability model generating unit 130, and a disease risk predicting unit 140, but is not limited thereto.

The information input unit 100 may receive a subject state variable of a subject and subject genetic information. In order to obtain the subject state variable of a subject, the information input unit 110 may provide a plurality of living condition variables and health condition variables to a user terminal. For example, in the user terminal, lists corresponding to the plurality of living condition variables and health condition variables are output and the user may input information corresponding to living condition variables and health condition variables.

According to an exemplary embodiment of the present disclosure, the state variables may be a living condition variable and a health condition variable of the subject including demographical characteristics such as an age, a gender, or a household income, epidemiological information such as a family history or a past disease history, a lifestyle such as a drinking history, a smoking history, a physical activity, or nutrition, physical measurement values such as a height, a weight, or a blood test result, and clinical information. The gene information may be genetic information collected in the form of a single nucleotide polymorphism.

The information input unit 110 may receive a subject state variable of a subject and subject genetic information from the disease prevention server 200. The disease prevention server 200 may provide generic data source of Ansan-Anseong Cohort which is a part of Korean Genome and Epidemiology Study performed by Korea Centers for Disease Control and Prevention and follow-up data obtained through first to seventh tracking as the subject state variable and the subject gene information, but is not limited thereto.

The machine learning model generating unit 120 may have a plurality of state variables including a living condition variable and a health condition variable of a patient with a metabolic disorder, gene information, and a disease risk of metabolic disorders as inputs. For example, the patient with a metabolic disorder may be a patient with a disease such as hypertension, diabetes, obesity, and metabolic syndrome. The plurality of state variables of the patient with a metabolic disorder may be individual's lifestyle and health condition information which are repeatedly measured. The genetic information of the patient with a metabolic disorder may be data collected at a single timing of a baseline investigation. Genomes associated with diseases of metabolic disorders may be genomic information known through the reference literature. The machine learning model generating unit 120 may be supplied with the plurality of state variables, genetic information, and a disease risk of metabolic disorders of patients with metabolic disorders from the disease prediction server 200. The plurality of state variables and the genetic information of the patients with metabolic disorders supplied from the disease prediction server 200 may be seventh follow-up data obtained by periodic monitoring and whether the disease (for example, hypertension, diabetes, obesity, and metabolic syndrome) of the subject occurs may be confirmed using the genetic information and the follow-up data.

The machine learning model generating unit 120 mas generate a machine learning model which learns information of a relationship between at least one of the plurality of state variables and the genetic information and the disease risk of the metabolic disorders. For example, the machine learning model may be generated using a recurrent neural network (RNN) and a multi-layer perceptron neural network (MLP).

According to an exemplary embodiment of the present disclosure, the machine learning model generating unit 120 connects genes associated wish individual diseases of the metabolic disorders to the multi-layer perception neural network to be connected and input to the recurrent neural network. Further, the machine learning model generating unit 120 sequentially inputs a plurality of state variables which is repeatedly measured to analyze not only a correlation of each epidemiologic variable according to a time, but also a correlation between variables through the plurality of state variables.

The machine learning model generating unit 120 may repeatedly measure a subject state variable of the subject and genetic information of the subject and input the repeatedly measured information. The machine learning model generating unit 120 may confirm whether the lifestyle is changed, for repeatedly measured values such as a lifestyle, physical measurement values, and clinical values, based on the subject state variable and the genetic information of the subject. The machine learning model generating unit 120 classifies groups showing similar patterns among the repeatedly measured values to generate a cluster for each group and classifies groups showing a similar lifestyle change pattern for every gender and disease. The machine learning model generating unit 120 may select a significant gene related to the change of the lifestyle for each disease of metabolic disorders, based on the subject genetic information of the subject. The significant gene may be a gene associated with each disease of the metabolic disorders.

According to an exemplars embodiment of the present disclosure, the machine learning model generating unit 120 may sequentially input the subject state variables which are repeatedly measured to the recurrent neural network among artificial neural networks. Further, the machine learning model generating unit 120 may connect the significant gene related to the change of the lifestyles for every disease of metabolic disorders to the recurrent neural network through the multi-layer perceptron.

The machine model generating unit 120 may have time series data such as a plurality of state variables including the living condition variable and the health condition variable as inputs. Further, the machine learning model generating unit 120 applies the recurrent neural network among artificial neural networks to generate a machine learning model. The machine learning model generating unit 120 may additionally connect the multi-layer perceptron neural network to a last layer of the existing recurrent neural network to collectively input the genetic information collected at the single timing. The machine learning model generating unit 120 may set whether hypertension, diabetes, obesity, and metabolic syndrome occur to a last output layer.

For example, the artificial neural network may be divided into three layers of an input layer, a hidden layer, and an output layer, each layer is configured by nodes and the input layer receives input data from the outside of the system to transmit the input data to the system. The hidden layer is located inside the system and receives the input value to process the input data and then calculate a result. The output layer may calculate a system output value based on the input value and the current system state. The input layer may input values of prediction variables (input variables) for deriving a prediction value (an output variable). When there are n input values in the input layer, the input layer has n nodes. In the present disclosure, values which are input to the input laves may be a plurality of state variables including a living condition variable and a health condition and genetic information. The hidden layer receives the input values from a plurality of input nodes to calculate a weight sum and applies this value to a transfer function to transmit the value to the output layer. For example, the input layer of the machine learning model may be a plurality of status information, genetic information, the hidden layer at a previous timing, the hidden layer may be a plurality of status information and information obtained by grouping the plurality of status information, and the output layer may indicate a disease risk.

According to an exemplary embodiment of the present disclosure, when a first state variable among the plurality of state variables is assumed to be an input layer and a second state variable among the plurality of state variables is assumed to be a hidden layer, the machine learning model performs first learning to learn information of a relationship between the input layer and the hidden layer. Further, when a previous state variable of the plurality of state variables is assumed to be an input layer and a current state variable of the plurality of state variables is assumed to be a hidden layer, the machine learning model may perform first learning to learn information of a relationship between the input layer and the hidden layer.

The machine learning model may learn a degree of a relationship between the input layer and the hidden layer, based on Equation 1. The degree of the relationship may be a value obtained by calculating a weight sum of information input to the input layer, but is not limited thereto.

hₜ=tan h(Wₕₕhₜ₋₁+Wₓₕxₜ) [Equation 1]

In this case, hₜ is a hidden layer at a liming t, hₜ₋₁ is a hidden layer at a previous timing of the timing t, xₜ is a first state variable. Wₕₕ is a first weight representing a degree of a first type of relationship between the input layer and the hidden layer, and Wₓₕ is a second weight representing a degree of a second type of relationship between the input layer and the hidden layer. For example, in Equation 1, xₜ is a first state variable among a plurality of state variables, hₜ is a hidden layer at a timing t, Wₓₕ is a weight between the plurality of state variables (input variables) and the hidden layer, and Wₕₕ may be a weight between hidden layers, but they are not limited thereto. For example, the degree of the first type of relationship may be a correlation (weight) between the plurality of state variables according to a time and the degree of the second type of relationship may be a correlation (weight) between the plurality of state variables, but they are not limited thereto.

The machine learning model inputs a plurality of state variables (for example, individual's lifestyle and health condition variable) which is repeatedly measured to the recurrent neural network represented in Equation 1 to analyze not only the correlation according to the time, but also the correlation between the lifestyle and the health condition variable.

According to the exemplary embodiment of the present disclosure, when the hidden layer and the genetic information are assumed as the input layer and the disease risk is assumed as the output layer, the machine learning model may perform the second learning to learn the information of the relationship between the hidden layer and the output layer. Further, when the hidden layer and the genetic information are assumed as the input layer and the disease risk is assumed as the output layer, the machine learning model may perform the second learning to learn the information of the relationship between the hidden layer and the output layer.

The machine learning model may learn a degree of a relationship between the hidden layer and the output layer, based on Equation 2. The second learning may learn a degree of a relationship between the hidden layer and the output layer, based on Equations 1 and 2. The machine learning model may learn information of the relationship between the input layer, the hidden layer, and the output layer based on Equations 1 and 2 and learn the prediction result of the disease risk as a result of the output layer.

y=sigmoid(W_{yh}hₜ+W_{yz}z) [Equation 2]

In this case, y is the output layer, W_{yh} is a third weight representing a degree of the relationship between the hidden layer and the output layer, hₜ is a hidden layer, W_{yz} is a fourth weight representing a degree of the relationship between the genetic information of the input layer and the output layer, and z is the genetic information of the input layer. For example, the third weight may be a degree of the relationship representing a relationship between the plurality of state variables and the output layer to predict the disease risk and the fourth weight may be a degree of the relationship between the genetic information and the output layer to assign a weight to a specific gene.

According to an exemplars embodiment of the present disclosure, since the genetic information is collected at the single timing, in order to combine the genetic information to the recurrent neural network, as represented in Equation 2, the genetic information may be input by connecting the multi-layer perceptron neural network to the last layer of the recurrent neural network. For example, the genetic information is collected in the form of single nucleotide polymorphism and known genetic information may be converted into a risk factor according to an allele to input the known genetic information for every metabolic disorder (hypertension, diabetes, obesity, and metabolic syndrome). The machine learning model may learn the degree of the relationship between the hidden layer and the output layer, that is, the weight between the hidden layer and the output layer, through the second learning.

According to an exemplary embodiment of the present disclosure, the machine learning model generating unit 120 may update the weight to an error generated when the machine learning model to learn a degree of the relationship between at least one of the plurality of state variables and genetic information and the disease risk of the metabolic disorders is generated based on Equation 3.

E=(t−y)²+λ∥W∥₂² [Equation 3]

Here, E is a detected error of the machine learning model generating unit 120, t is whether the metabolic disorder occurs, y is a disease risk predicted through a machine learning model, and ∥W∥₂² is an L2 regular expression for preventing overfitting due to the error.

Equation 3 is an error equation of the machine learning model generating unit 120 and learns a weight of the artificial neural network through a backpropagation algorithm. In order to prevent overfitting due to the noise generated during the learning process, the L2 regular expression is added and t indicates whether an actual metabolic disorder (hypertension, diabetes, obesity or metabolic syndrome) occurs, but is not limited thereto.

According to an exemplary embodiment of the present disclosure, the machine learning model generating unit 120 divides patients (all subjects) with the metabolic disorders into three groups to verify the specificity(validity) of the constructed machine learning model (for example, the artificial neural network) to perform cross validation. The machine learning model generating unit 120 adjusts a weight for the plurality of state variables including the living condition variable and the health condition variable associated with the generation of the metabolic disorder (hypertension, diabetes, obesity, and metabolic syndrome) through the literature review after validation to generate a solid machine learning model.

According to an exemplary embodiment of the present disclosure, the disease risk predicting unit 140 applies the subject state variable and the subject genetic information to the machine learning model to predict the subject disease risk of the subject.

According to an exemplary embodiment of the present disclosure, the statistical probability model generating unit 130 may include a basic statistical probability model generating unit 131 and a weight statistical probability model generating unit 132.

The statistical probability model generating unit 130 may generate a statistical probability model probabilistically representing the disease risk of the metabolic disorders depending on whether there are at least one of the plurality of state variables and genetic information or the value, with the plurality of state variables, the genetic information, and the disease risk of the metabolic disorder of the patient with the metabolic disorder as an input. For example, the statistical probability model generating unit 130 may identify a group to which the subject belongs, among currently divided four groups (low, normal, high, very high). Further the statistical probability model generating unit 130 may predict a risk R of a disease observed tor every subject and a risk Ro of a disease expected for every variable combination representing a baseline risk, based on an influence degree b of the disease risk for every variable (a plurality of state variables) and finally calculate a unique risk score of each subject using them.

According to an exemplary embodiment of the present disclosure, the basic statistical probability model generating unit 131 inputs the plurality of state variables the genetic information, and the disease risk of the metabolic disorders of the patient with the metabolic disorder selects at least one variable associated with the metabolic disorder among the plurality of state variables, and generates the basic statistical probability model probabilistically representing the disease risk of the metabolic disorder for whether there is at least one state variable or the value.

For example, the basic statistical probability model generating unit 131 may input a plurality of state variables (for example, repeatedly measured information of a factor such as a lifestyle, a physical measurement value, or a disease history) which may be recognized by the individual (the subject or the patient). Further, the basic statistical probability model generating unit 131 may generate a statistical probability model probabilistically representing a disease risk of the metabolic disorder based on the first to seventh follow-up data of Ansan-Anseong Cohort which is a part of Korean Genome and Epidemiology Study performed by Korea Centers for Disease Control and Prevention supplied from the disease prediction server 200 . Further, the statistical probability model generating unit 130 may generate a statistical probability model probabilistically representing the disease risk of the metabolic disorder based on an input for individual's lifestyle and health condition information at the time of baseline investigation. Further, the basic statistical probability model generating unit 131 may select a major variable based on the statistical probability model probabilistically representing the disease risk of the metabolic disorder for a repeatedly measured value for a factor such as nutrition and clinical values which cannot be recognized by the individual.

The basic statistical probability model generating unit 131 may primarily select a major variable using the statistical probability based model among the plurality of state variables which is recognizable by the individual. The basic statistical probability model generating unit 131 may secondarily select a major variable using the statistical probability based model based on a factor such as nutrition and clinical values which cannot be recognized by the individual. Further, the basic statistical probability model generating unit 131 may select a major variable for a bask statistical probability model probabilistically representing the disease risk of the metabolic disorders based on the primary and secondary major variables selection. For exemplary, the above-described statistical probability model may select a primary variable (major variable) for variables which are selected two times or more through three variable selecting processes of a forward selection method, a backward selection method, and a stepwise entry method using a Cox proportional hazards regression model which is one of methods of a statistical probability model.

Further, the basic statistical probability model generating unit 131 may additionally select a variable associated with each disease of the metabolic disorder on the medical and clinical basis. According to the gene selection based on the genetic information, a significant gene for every disease of the metabolic disorders based on the input genetic information is selected first and a gene which is not statistically significant, but is reported to have a correlation with the existing disease is additionally selected to finally select a gene. Further, the basic statistical probability model generating unit 131 may finally select a variable included in disease prediction of the metabolic disorder by additionally inputting the clinically significant variable under a medical judgment of experts.

Further, the basic statistical probability model generating unit 131 may divide the subjects into a training set and a test set at a ratio of 7 to 3 for model construction and validation. The basic statistical probability model generating unit 131 may generate a basic statistical probability model for predicting obesity, prehypertension, prediabetes associated with the current metabolic disorder of the subject using a statistical model based competitive probability risk model in the training set using the selected variable. The basic statistical probability model generating unit 131 may extract an optimal value for an influence b to the disease occurrence for every variable (each of the plurality of state variables) through internal validation and five-fold cross validation in the test set and generate a basic statistical probability model of the final disease occurrence using the same.

The weight statistical probability model generating unit 132 applies a weight to the disease risk of the metabolic disorder depending on whether there is genetic information associated with the metabolic disorder to generate a statistical probability model from the basic statistical probability model.

According to an exemplary embodiment of the present disclosure, the statistical probability model generating unit 130 may generate a statistical probability model which probabilistically represents a disease risk of the hypertension depending on whether there is at least one of the plurality of state variables and genetic information or a value. For example, the statistical probability model generating unit 130 may select clinically relevant variables (for example, a family history, a past disease history, an age, a gender, eating habits, and a lifestyle) for the current prehypertension and prevalence prediction of the hypertension. The statistical probability model generating unit 130 may select a risk factor for the hypertension prevalence state by sequentially applying univariate and multivariate logistic models and finally select 24 variables through the backward selection method.

The statistical probability model generating unit 140 may calculate a prevalence probability of the prehypertension based on Equation 4.

Prehypertension Ps=1/(1+e^{b1}) [Equation 4]

According to an exemplary embodiment of the present disclosure, b1 may be a weight applied to the disease risk of the metabolic disorder depending on whether there are at least one selected state variable relevant to the metabolic disorder among a plurality of state variables relevant to the prehypertension and genetic information relevant to the metabolic disorder.

b1(prehypertension)=(0.37156*[age=50-59]+0.80200*[age=60-69]+0.89609*[age=70+]−0.41552*[gender=female]+0.43825*[education level=uneducated]+0.32208*[education level=elementary school]+0.19062*[education level=middle school]+0.13103*[education level=high school]−0.03046*[education level=four-year-course college]+0.11333*[monthly average income=less than three million won]+0.05827*[monthly average income=300-399]−0.13926*[monthly average income=six million won+]+0.23111*[ALT=20-39]+0.43178*[ALT=40+]−0.12783*[Hb=anemia]+0.34359*[Hb male 15/female 14 or higher]+0.32334*[proteinuria=2+−4+]+0.06766*[glucose in urine=+−−1+]+0.27763*[glucose in urine=2+−4+]+0.18232*[total cholesterol=200-239]+0.30748*[total cholesterol=240+]+0.17395*[HDL=less than 40]+0.12222*[HDL=40-59]+0.06766*[sodium intake=excessive]+0.00995*[potassium intake=excessive]+0.00995*[protein intake=sufficient, fat intake=excessive]−0.05129*[drinking status=stop drinking]+0.10436*[drinking status=current drinking]+0.01980*[passive smoking=yes]+0.21511*[hyperlipidemia=yes]+0.04879*[angina pectoris=yes]+0.15700*[fatty liver=yes]−0.13926*[allergies=yes]+0.04879*[arthritis=yes]+0.13976*[hscrp=0.3+]−0.12783*[uric acid level in blood=moderate]+0.25464*[uric acid level in blood=high]+0.37844*[family history of metabolic disorder=1]+0.37844*[family history of metabolic disorder=2 or more]+0.02956[exercise enough to sweat=5+times/week]

Further, the statistical probability model generating unit 140 may calculate a prevalence probability of the hypertension based on Equation 5.

Hypertension P=1/(1+e^{b2}) [Equation 5]

According to an exemplary embodiment of the present disclosure, b2 may be a weight applied to the disease risk of the metabolic disorder depending on whether there is at least one selected state variable relevant to the metabolic disorder among a plurality of state variables relevant to the hypertension and genetic information relevant to the metabolic disorder.

b2 (hypertension)=(0.60432*[age=50-59]+1.26695*[age=60-69]+1.51732*[age=70+]−−0.49430*[gender=female]+0.77932*[education level=uneducated]+0.51879*[education level=elementary school]+0.31481*[education level=middle school]+0.19062*[education level=high school]−0.04082*[education level=four-year-course college]+0.23111*[monthly average income=less than three million won]+0.08618*[monthly average income=300-399]−0.16252*[monthly average income=six million won+]+0.37156*[ALT=20-39]+0.70310*[ALT=40+]−0.16252*[Hb=anemia]+0.58222*[Hb male 15/female 14 or higher]+0.29267*[proteinuria=+]+1.13140*[proteinuria=2+−4+]+0.30010*[glucose in urine=+]+0.58222*[glucose in urine=2+−4+]0.2858*[total cholesterol=200+]+0.46373*[total cholesterol=240+]+0.16551*[HDL=less than 60]+0.07696*[sodium intake=excessive]+0.09531*[potassium intake=excessive]−0.04082*[protein intake or fat intake=above one reference value]−0.09431*[protein intake=sufficient fat intake=excessive]−0.10536*[drinking state=stop drinking]+0.19885*[drinking state=currently drinking]+0.11333*[passive smoking=yes]+0.23111*[hyperlipidemia=yes]+0.18232*[fatty liver=yes]−0.21072*[allergic disease=yes]+0.10436*[arthritis=yes]+0.25464*[hscrp=0.3+]−0.16252*[uric acid level in blood=low]+0.62594*[uric acid level in blood=high]+0.40547*[family history of metabolic disorder=one]+0.61519*[family history of metabolic disorder=2 or more]+0.07696[exercise enough to sweat=5+times/week])

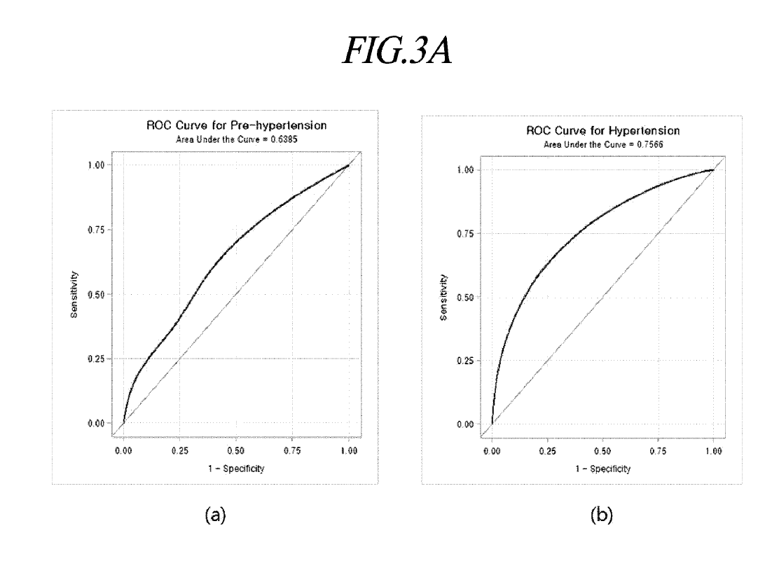

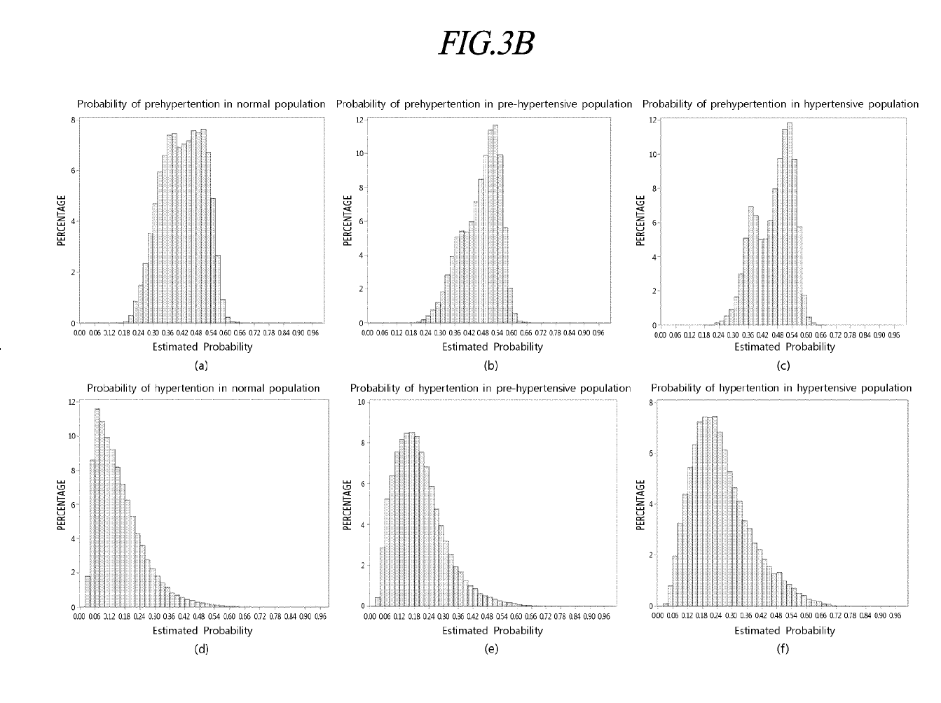

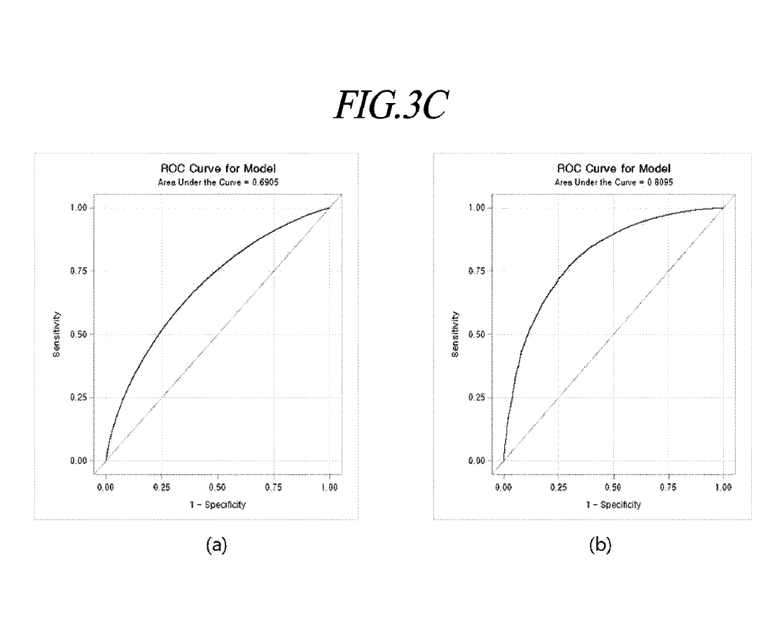

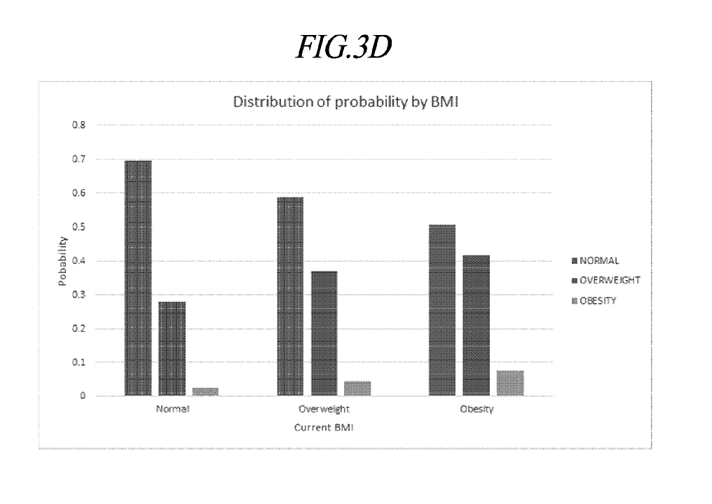

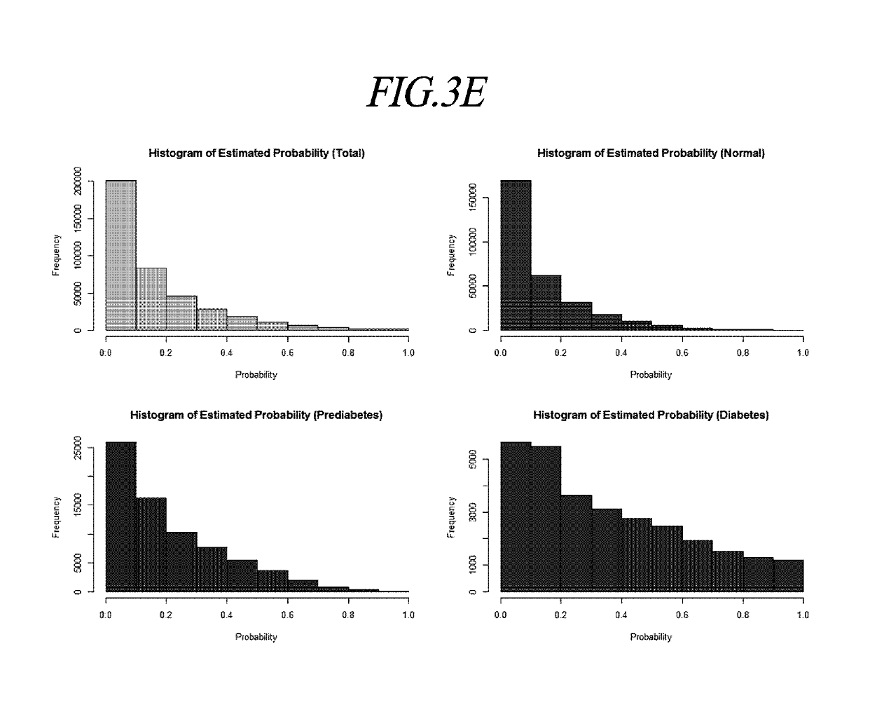

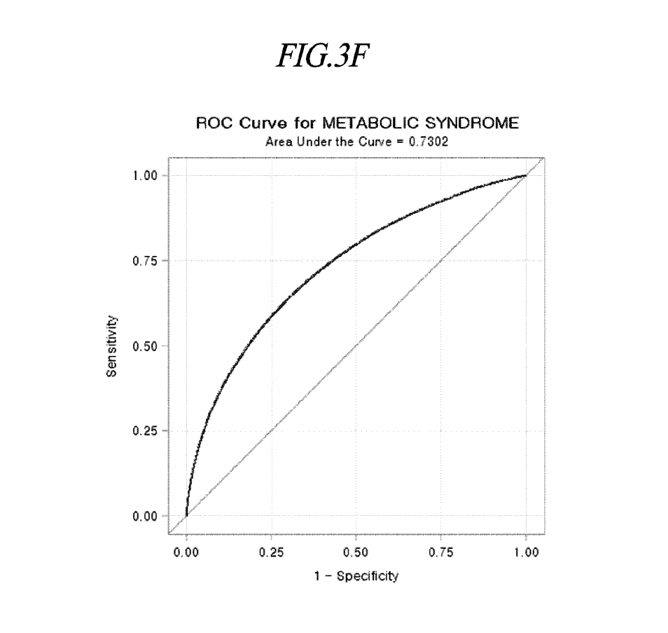

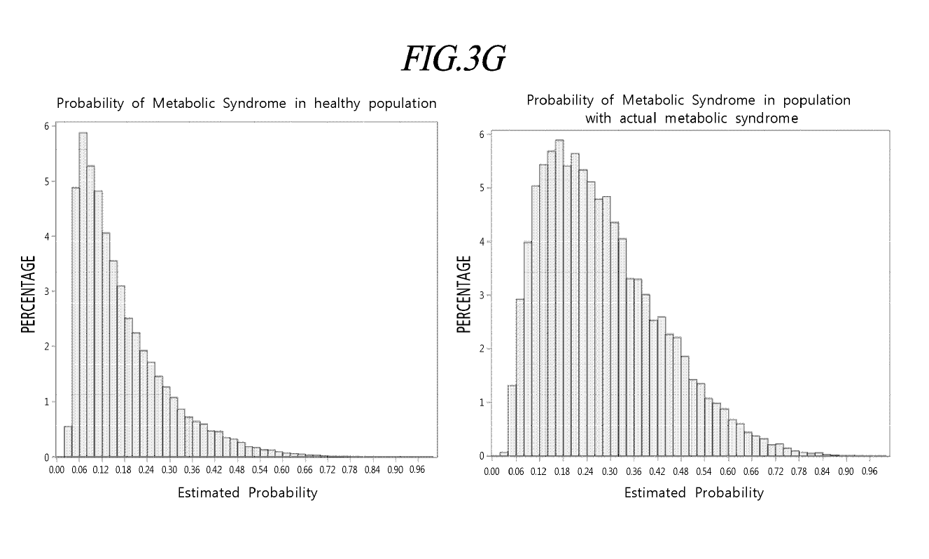

FIG. 3A may be a graph illustrating a prehypertension prediction ROC curve and a hypertension prediction ROC curve. For example, referring to FIG. 3A, the statistical model generating unit 130 may perform internal validation test to evaluate a prediction ability of a prevalence probability prediction model. In (a) of FIG. 3A, a c-statistic (95% confidence interval) of the prehypertension prediction model is calculated to be 0.639 (0.635-0.042) and in (b) of FIG. 3A, c-statistic (95% confidence interval) of the hypertension prediction model is calculated to be 0.757 (0.7.54-0.760).

Referring to FIG. 3A, it is possible to confirm a distribution according to the current normal, prehypertension, and hypertension states of the prehypertension and the hypertension probabilities predicted through the constructed final prediction model. It is confirmed that the probability of prehypertension and the probability of hypertension of the subject for the prehypertension and the hypertension are increasing through the constructed final prediction model.

FIG. 3B is a graph illustrating a probability distribution in prehypertension and hypertension groups. For example, referring to FIG. 3B, (a) of FIG. 3B is a graph illustrating a probability distribution of prehypertension in a healthy weight population. (b) is a graph illustrating, a probability distribution of prehypertension in a pre-hypertensive population, (c) is a graph illustrating a probability distribution of prehypertension in a hypertensive population, (d) is a graph illustrating a probability distribution of hypertension in the healthy weight population, (e) is a graph illustrating a probability distribution of hypertension in a pre-hypertensive population, and (f) is a graph illustrating a probability distribution of hypertension in a hypertensive population.

Further, according to an exemplary embodiment of the present disclosure, the statistical probability model generating unit 130 may generate a statistical probability model which probabilistically represents a disease risk of the obesity depending on whether there is at least one of the plurality of state variables and genetic information or a value. For example, the statistical probability model generating unit 130 may select relevant variables (for example, a family history, a past history, an age, a gender, eating habits, and a lifestyle known by existing studies for the current overweight and prevalence prediction of the obesity. The statistical probability model generating unit 130 may select a risk factor for the hypertension prevalence state by sequentially applying univariate and multivariate logistic models and finally select 24 variables through the backward selection method.

The statistical probability model generating unit 140 may calculate a prevalence probability of the overweight based on Equation 6.

Overweight Ps=1/(1+e^{b3}) [Equation 6]

According to an exemplary embodiment of the present disclosure, b3 may be a weight applied to the disease risk of the metabolic disorder depending on whether thee are at least one selected state variable relevant to the metabolic disorder among a plurality of state variables relevant to the overweight and genetic information relevant to the overweight.

b3(overweight)=(−0.02020*[age=50-59]−0.01005*[age=60-69]−0.18633*[age=70+]−0.05129*[gender female]+0.50683*[education level=uneducated]+0.32930*[education level=elementary school]+0.50682*[education level=middle school]+0.01980*[education level=high school]+0.19062*[education level=four-year-course college]+0.18232*[past history of hyperlipidemia=yes]+0.23902*[past history of myocardial infarction=yes]+0.62594*[past history of fatty liver=yes]+0.13976*[past history of cholecystitis=yes]−0.10536*[past history of allergy=yes]−0.10536*[thyroid gland disease=yes]+0.29267*[arthritis=yes]+0.47623*[blood pressure=hypertension stage 1]+0.62058*[blood pressure=hypertension stage 2]+0.06766[exercise enough to sweat=no]−0.03046 [excessive enough to sweat=5+ time/week]+0.07696*[daily average energy intake excessive]+0.02956*[sodium intake compared with energy intake=intermediate]+0.07696*[sodium intake compared with energy intake=high]+0.11333*[protein intake or fat intake=above one reference value]+0.20701*[protein intake=sufficient, fat intake=excessive]+0.55389*[ALT=20-39]+0.94001*[ALT=40+]−0.10536* [Hb=anemia]+0.25464*[Hb male 15/female 14 or higher]+0.12222*[proteinuria=1+]+0.17395*[proteinuria=2+−4⇄]+0.23111*[total cholesterol=200-239]+0.39204*[total cholesterol=240+]+1.02962*[HDL=less than 40]+0.61519*[HDL=40-59]+0.30010*[fasting blood sugar=110-125]+0.23902*[fasting blood sugar=126+]−0.05129*[drinking state=stop drinking]+0.10436*[drinking state=currently drinking]+0.01980*[passive smoking=yes]+0.37844*[hscrp=0.3-0.99]+0.08618*[hscrp=1.0+]−0.35667*[uric acid level in blood=moderate]+0.48858*[uric acid level in blood=high]+0.05827*[family history of metabolic disorder=1]+0.11333*[family history of metabolic disorder=2 or more])

The statistical probability model generating unit 130 may calculate a prevalence probability of the obesity based on Equation 7.

Obesity Ps=1/(1+e^{b4}) [Equation 7]

According to an exemplary embodiment of the present disclosure, b4 may be a weight applied to the disease risk of the metabolic disorder depending on whether there are at least one selected state variable relevant to the metabolic disorder among a plurality of state variables relevant to the obesity and generic information relevant to the metabolic disorder.

b4(obesity)=(−0.35667*[age=50-59]−0.52763*[age=60-69]−0.73397*[age=70+]+0.84157*[gender=female]+0.63127*[education level=uneducated]+0.33647*[education level=elementary school]+0.05827*[education level=middle school]+0.07696*[education level=high school]+0.14842*[education level=four-year-course college]+0.33647*[past history of hyperlipidemia=yes]+0.62594*[past history of myocardial infarction=yes]+0.87547*[past history or fatty liver=yes]+0.30010*[past history of cholecystitis=yes]−0.18633*[past history or allergy=yes]−0.22314*[thyroid gland disease=yes]+0.62058*[arthritis=yes]+0.93216*[pressure=hypertension stage 1]+1.24415*[pressure=hypertension stage 2]+0.21511 [exercise enough to sweat=no]+0.11333*[exercise enough to sweat=5+ times/week]+0.113333*[daily average energy intake=excessive]+0.07696*[sodium intake compared with energy intake=intermediate]+0.16551*[sodium intake compared with energy intake=high]+0.21511*[protein intake or fat intake=above one reference value]+0.47000*[protein intake=sufficient, fate intake=excessive]+1.02962*[ALT=20-39]+1.93297*[ALT=40+]−0.04082*[Hb=anemia]+0.36464*[Hb male 15/female 14 or higher]+0.35066*[proteinuria=1+]+0.54812*[proteinuria=2+−4+]+0.22314*[total cholesterol=200-239]+0.37156*[total cholesterol=240+]+1.32442*[HDL=less than 40]+0.76547*[HDL=40-59]+0.71295*[fasting blood sugar=110-125]+0.63127*[fasting blood sugar=126+]−0.05129*[drinking state=stop drinking]+0.10436*[drinking state=currently drinking]+0.01980*[passive smoking=yes]+1.05779*[hscrp=0.3-0.99]+0.57661*[hscrp=1.0+]−0.69315*[uric acid level in blood=moderate]+0.90826*[uric acid level in blood=high]+0.08618*[family history of metabolic disorder=1]+0.23902*[family history of metabolic disorder=2 or more]).

FIG. 3C is a view schematically illustrating an overweight and obesity prediction ROC curve. For example, referring to FIG. 3C, the statistical model generating unit 130 may perform an internal validation test to evaluate a prediction ability of a prevalence probability prediction model. In (a) of FIG. 3C, a c-statistic (95% confidence interval) of an overweight prediction model is calculated to be 0.691 (0.688-0.693) and in (b) of FIG. 3C, a c-statistic (95% confidence interval) of the hypertension prediction model is calculated to be 0.810 (0.804-0.815). Referring to the graph of FIG. 3C, an explanatory power of the obesity prediction model was higher as compared with the weight and in the case of obesity, the distribution of the risk factor of the healthy population is more obvious than the overweight population.

Referring to FIG. 3C, it is possible to confirm a distribution according to the current normal, overweight, and obesity states of the overweight and obesity probabilities predicted through the constructed final prediction model. It is confirmed that both the probability of overweight and. the probability of obesity of the subject for the overweight and obesity are increasing.

FIG. 3D is a probability distribution graph of normal, overweight, and obesity prediction according to the current normal, overweight, and obesity states for example, referring to FIG 3D, it is confirmed that the graph illustrated in FIG. 3D illustrates a normal overweight and obesity prediction probability distributions according to the current normal, overweight and obesity states.

According to an exemplary embodiment of the present disclosure, b4 may be a weight applied to a disease risk of the metabolic disorder depending on there are at least one selected state satiable relevant to the metabolic disorder among a plurality of state variables relevant to the obesity and genetic information relevant to the metabolic disorder. For example, the statistical probability model generating unit 140 selects 120 variables which have a missing value for diabetes which does not exceed 20% and are clinically significant and reconstructs continuous variables as categories according to quartiles to generate a statistical model. The statistical probability model generating unit 140 selects a risk factor for a prevalence state of the diabetes by applying an automated forward selection method, backward selection method, and stepwise selection method for variable selection of a multivariate logistic model and calculates a c-statistic of the result model to select a stepwise selection model formed of 65 variables which is determined to have the highest explanatory power as a final model.

The statistical probability model generating unit 140 may calculate a prevalence probability of the diabetes based on Equation 8.

Diabetes Ps=1/(1+e^{b5}) [Equation 8]

b5(diabetes)=(−0.04082*[education level=middle/high school]−0.18633*[education level=two/four-year-course college]−0.07257*[marriage state=married]0.01980*[occupation=office job]+0.07696*[occupation=housewife]0.05827*[occupation=others]+0.02956*[income=2Q]−0.08338*[income=4Q]+0.54232*[gender=female]+0.02956*[full age (continuous)]+0.36464*[past history of hypertension=yes]+0.14842*[past history of hyperlipidemia=yes]+0.14842*[past history of myocardial infarction=yes]−0.19845*[past history of chronic gastritis=yes]+0.16551*[past history of fatty liver=yes]+0.11333*[past history of colecystitis=yes]−0.17435*[past history of chronic bronchitis=yes]−0.10536*[past history of asthma=yes]−0.18633*[past history of allergy=yes]−0.16252*[arthritis=yes]−0.19845*[past history of osteoporosis=yes]+0.21511*[past history of cataract=yes]−0.10536*[past history of depressive disorder=yes]−0.03046*[past history of thyroid gland disease=hyperthroidism]−0.21072*[past history of thyroid gland disease=hypothryoidism]−0.05129*[past history of thyroid gland disease=others]+0.07696*[number of exposure to passive smoking=upper 50%]+0.04879*[number of passive smoking exposure=lower 50%]−0.01005*[total alcohol intakes=1Q]+0.04879*[total alcohol intakes=2Q]+0.17395*[total alcohol intakes=3Q]+0.12222 [number of exercises=upper 50%]−0.04082*[age of first birth=2Q]−0.08338*[age of firth birth=3Q]−0.06188*[age of first birth=4Q]+0.90016*[past history of gestational diabetes=yes]−0.05129*[past history of induced abortion=yes]+0.26236*[past history of birth of fetal macrosomia=yes]+0.02956*[whether to take oral contraceptive pill=taken in the past]−0.32850*[whether to take oral contraceptive pill=currently taking]+0.06766*[family history of diabetes=yes]−0.07257*[family history of angina pectoris=yes]−0.08338*[family history of stroke=yes]−0.12222*[current subjective health condition=4 points]+0.19062*[current subjective health condition=3 points]+0.39878*[current subjective health condition=2 points]+0.48858*[current subjective health condition=1 point]+0.03922*[“I am very comfortable and feel healthy now”=3 points]+0.08618*[“I am very comfortable feel healthy now”=2 points]+0.12222*[“I am very comfortable and feel healthy now”=1 Point]−0.09431*[“I do not feel refreshed after sleep”=no]−0.10536*[“I do not feel refreshed after sleep”=yes]−0.03046*[“I do not feel refreshed after sleep”=very true]−0.01005*[“I feel full of energy (vigor).”=3 points]−0.04082*[“I feel full of energy (vigor).”=2 points]−0.09431*[“I feel full of energy (vigor).”=1 point]+0.01980* [“I become upset or anxious at night.”=3 points]−0.05129*[“I become upset or anxious at night.”=1 point]−0.24846*[hematuria=4Q]−0.28768*[hematuria=3Q]−0.487804* [hematuria=2Q]+0.17395*[ALT=20-39]+0.41871*[ALT=40+]−0.11653*[Hb=anemia]−0.08338*[Hb=normal]−0.02020*[fat(g)]−0.01005*[carbohydrate(g)]+0.00995*[iron(mg)]+0.25465*[vitamin B1(mg)]+0.00995*[zinc(ug)]−0.21072*[vitamin B6(mg)]+0.01980*[weight]+0.02956*[waist size]−0.13926*[hip circumference=2Q]−0.24846*[hip circumference=3Q]−0.40048*[hip circumference=4Q]+0.09531*[pulse rate=2Q]+0.23902*[pulse rate=3Q]−0.41471*[pulse rate=4Q]+0.14842*[diastolic blood pressure=2Q]+0.27763*[systolic blood pressure=3Q]+0.41211*[diastolic blood pressure=4Q]+0.03922*[diastolic blood pressure=2Q]−0.02020*[diastolic blood pressure=3Q]−0.11653*[diastolic blood pressure=4Q]+0.19062*[γ-GTP=2Q]+0.43178*[γ-GTP=3Q]+0.63658*[γ-GTP=4Q]+0.14842*[Albumin=2Q]+0.27003*[Albumin=3Q]+0.48858*[Albumin=4Q]+0.03922*[BUN=2Q]+0.13103*[BUN=3Q]+0.23902*[BUN=4Q]−0.12783*[Creatinine]−0.04082*[Uric Acid=2Q]−0.05129*[Uric Acid=3Q]−0.19845*[Uric Acid=4Q]−0.13926*[Total cholesterol=2Q]−0.13926*[Total cholesterol=3Q]−0.08338*[Total cholesterol=4Q]]−0.01005*[HDL−cholesterol=2Q]−0.07257*[HDL−cholesterol=3Q]−0.08338*[HDL−cholesterol=4Q]]+0.16551*[Triglyceride=2Q]+0.25464*[Triglyceride=3Q]+0.41871*[Triglyceride=4Q]]+0.04879*[body mass index=2Q]+0.10436*[body mass index=3Q]+0.09531*[body mass index=4Q])

The statistical probability model generating unit 140 may perform art internal validation test to evaluate a predict ton ability of the prevalence probability prediction model. The statistical probability model generating unit 140 may calculate a c-statistic (95% confidence interval) of the overweight prediction model to be 0.749.

FIG. 3E is a graph illustrating a probability of diabetes among metabolic disorders predicted by a multinomial logistic model obtained by the stepwise selection method. For example, referring to FIG. 3E, the distribution according to the current normal, prediabetes, and diabetes predicted through the constructed final prediction model may be confirmed. From the graph of FIG. 3E, it is confirmed that both the overweight probability and the obesity probability of the subjects of the prediabetes and the diabetes are increasing.

According to an exemplary embodiment of the present disclosure, the statistical probability model generating unit 130 may generate a statistical probability model which probabilistically represents a disease risk of the metabolic disorders depending on whether there is at least one of the plurality of state variables and genetic information or a value. For example, the statistical probability model generating unit 130 may select clinically relevant variables (for example, a family history, a past history, an age, a gender, eating habits, and a lifestyle) for the current metabolic syndrome. The statistical probability model generating unit 130 may select a risk factor for a metabolic syndrome prevalence slate by sequentially applying univariate and multivariate logistic models and finally select 21 variables through the backward selection method.

The statistical probability model generating unit 140 may calculate a prevalence probability of the metabolic syndrome based on Equation 9.

Metabolic syndrome Ps=/(1+e^{b6}) [Equation 9]

According to an exemplary embodiment of the present disclosure, b5 may be a weight applied to a disease risk of the metabolic disorder depending on there are at least one selected state variable relevant to the metabolic disorder among a plurality of state variables relevant to the metabolic syndrome and genetic information relevant to the metabolic disorder.

b5(metabolic syndrome)=(0.37156*[age=50-59]+0.77011*[age=60-69]+0.77932*[age=70+]+0.19062*[gender=female]+0.55962*[education level=uneducated]+0.29267*[education level=elementary school]+0.13976*[education level=middle school]+0.15700*[education level=high school]−0.01005*[education level=four-year-course college]+0.15700*[monthly average income=less than 3 million won]+0.06766*[monthly average income=300-399]−0.04082*[monthly average income=6 million won]+0.70804*[ALT=20-39]+1.28371*[ALT=40+]−0.11653*[Hb=anemia]+0.41211*[Hb male 15/female 14 or higher]+0.45108*[proteinuria=+]+1.12817*[proteinuria=2+−4−]+0.07696*[sodium intake=excessive]+0.12222*[potassium intake=excessive]+0.06766*[energy intake=excessive]+0.06766*[exercise enough to sweat=barely]+0.02956*[exercise enough to sweat=5+ times/week]+0.15700*[pack-years of smoking <20 PY]+0.30010*[pack-years of smoking=20-39]+0.27003*[pack-years of smoking=40+]+0.27763*[past history of myocardial infarction=yes]+0.57098*[past history of fatty liver=yes]+0.11333*[past history of cholecystitis=yes]−0.17435*[allergic disease=yes]−0.12783*[a past history of thyroid gland disease=yes]+0.10436*[arthritis=yes]+0.59333*[hscrp=0.3+]+0.20701*[hscrp=0.3+]−0.28768*[uric acid level in blood=low]+0.84157*[uric acid level in blood=high]+0.24686*[family history of metabolic disorder=1]+0.34359*[family history of metabolic disorder=2 or more])

FIG. 3F is a view schematically illustrating a metabolic syndrome prediction ROC curve. For example, referring to FIG. 3F, it is confirmed that a c-statistic (95% confidence interval) of the prevalence probability of the metabolic syndrome finally selected based on the statistical probability model generating unit 130 is 0.730 (0.728-0.733).

FIG. 3G is a graph schematically illustrating a metabolic syndrome probability distribution in a healthy population and a population with metabolic syndrome. (a) as of FIG. 3G is a probability distribution of metabolic syndrome in the healthy population and (b) is a graph illustrating a probability distribution of metabolic syndrome in the population with metabolic syndrome. Referring to FIG. 3G, the distribution of the metabolic syndrome prevalence probability predicted through the final prediction model constructed in the statistical probability model generating unit 130 depending on the current healthy state and the metabolic syndrome state may be confirmed. Further, referring to the graph illustrated in FIG 3G, it is confirmed that the probability value of the prevalence state of the actual metabolic syndrome is increased in a group with the metabolic syndrome.

According to an exemplary embodiment of the present disclosure, the disease risk predicting unit 140 applies the subject state variable and the subject genetic information to the machine learning model and the statistical probability model to predict the subject disease risk of the subject. Further, the disease risk predicting unit 140 may visualize the disease risk prediction result of the subject based on a predetermined classification category. For example, the disease risk predicting unit 140 constructs a deep-learning based visualization algorithm to provide a result visualized for each subject based on the machine learning model of the machine learning model generating unit 120 and the statistical probability model of the statistical probability model generating unit 130. The disease ask predicting unit 140 predicts, visualizes, and provides changes of individual's disease risk path based on a changing pattern of a negative factor. Further, the disease risk predicting unit 140 may visualize and provide a safe path through which the individual's disease risk probability is reduced, based on a changing pattern of a positive factor. Furthermore, the disease risk predicting unit 140 comprehensively considers the changing pal terns of the negative factor and the positive factor to provide a personalized preventive management service model through a risk avoidance path guidance for the metabolic disorder, a final health condition such as cardiovascular disease and chronic heart disease, and death, based on the changing pattern of lifestyle of each subject.

For example, the disease risk predicting unit 140 re-inputs a plurality of status information (lifestyle and health condition information) of the subject (individual) which is repeatedly measured to the machine learning model generating unit 120 and the statistical probability model generating unit 130 later to identify the change of the epidemiologic variable according to the time and apply and calculate the changing rate to the prediction model to provide a health condition modification result according to the intermediate health care of the subject and a re-predicted disease occurrence risk based thereon.

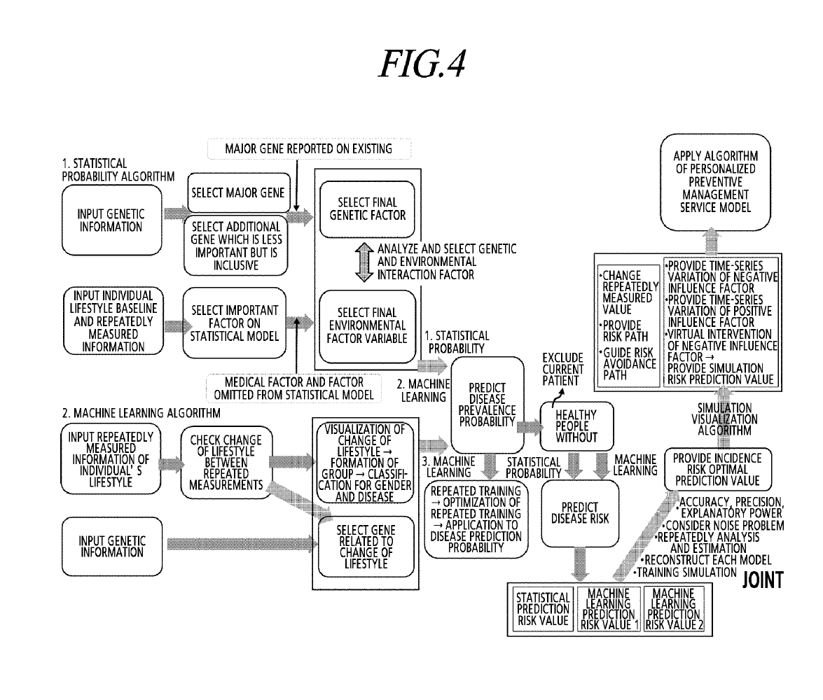

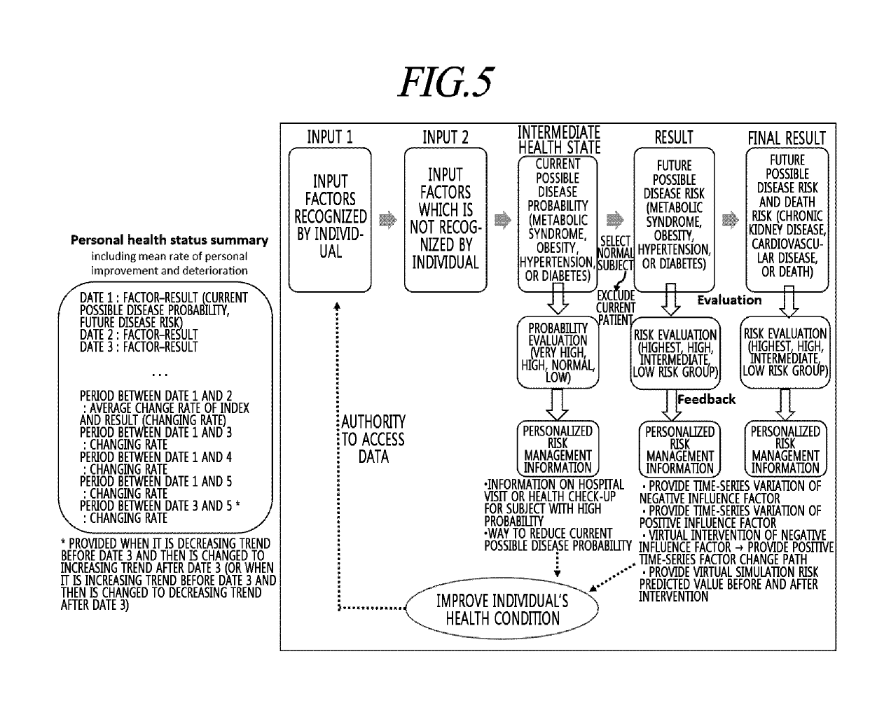

FIG. 4 is a view schematically illustrating a process of predicting a subject disease risk of a subject by applying a subject state variable of the subject and subject genetic information to a machine learning model and a statistical probability model according to an exemplary embodiment of the present disclosure. The process of predicting a subject disease risk described with reference to FIG. 4 is processed in each unit of the apparatus 100 for predicting a disease risk of metabolic disorders described with reference to FIGS. 1 to 3G. Therefore, even though it is not described below, the contents may be included in the description of the operation of the apparatus for predicting a disease risk of metabolic disorders described with reference to FIGS. 5 to 3G or deducible so that the detailed description will be omitted.

Referring to FIG. 4, 1. the statistical algorithm ma be a process performed by the statistical probability model generating unit 130. First, the statistical model generating unit 130 may have genetic information and an individual's lifestyle baseline and repeatedly measured information (a plurality of state variables) as inputs. The statistical model generating unit 130 may select a main gene based on the genetic information. In this case, an additional gene which is less important but is inclusive, may be selected among genes associated with the metabolic disorders. Further, the statistical model generating unit 130 may select an important gene associated with each metabolic disorder reported on the existing main research. The statistical model generating unit 130 may select a final gene associated with each metabolic disorder.

Next, the statistical model generating unit 130 may select an important status information factor on the statistical probability model associated with the metabolic disorder with a plurality of status information (individual's lifestyle baseline and repeatedly measured information) as inputs. The statistical model generating unit 130 may additionally select a medical factor and status information (factor) omitted from the statistical model. The statistical model generating unit 130 may select a plurality of final state variables (environmental factor variables).

The statistical model generating unit 130 probabilistically represents the disease risk of the metabolic disorders by applying a plurality of selected state variables. The statistical model generating unit 130 may predict the disease incidence risk by comparing a state variable of healthy people having, no disease with the statistical probability model of the plurality of state variables of the subject.

As illustrated in FIG. 4. 2. the machine learning algorithm may be a process performed by the machine learning model generating unit 120. The machine learning model generating unit 120 may have individual lifestyle repeatedly measured information (a plurality of state variables) as inputs. Further, the machine learning model generating unit 120 may have genetic information as an input. The machine learning model generating unit 120 may check the change between the plurality of repeatedly measured state variables. The machine learning model generating unit 120 may form a group of a plurality of similar state variables. The machine learning model generating unit 120 may divide the group of the plurality of similar state variables by gender and the metabolic disorders (hypertension, obesity, diabetes, and metabolic syndrome). The machine learning model generating unit 120 may select a significant gene related to the change of the lifestyle for every disease. The machine learning model generating unit 120 may optimize the prediction degree through repeated training of the machine learning model.

According to an exemplary embodiment of the present disclosure, the disease risk predicting unit 140 may visualize and provide the change between the plurality of repeatedly measured state variables The disease risk predicting unit 140 may provide an optimal prediction value among subject disease risk prediction values predicted based on the machine learning model generating unit 120 and the statistical probability model generating unit 130. For example, when a is determined that the prediction value predicted by the machine learning model with the plurality of state variables and genetic information of the subject as inputs is more precise than a prediction value generated based on the statistical model in the statistical probability model generating unit 130, the disease risk predicting unit 140 may provide the prediction value predicted by the machine learning model generating unit 120. The disease risk predicting unit 140 may provide a personalized preventive management service model by applying a simulation visualization algorithm. The disease risk predicting unit 140 may provide repeatedly measured (measurement value obtained by repeatedly measuring the plurality of status information) value change, the risk path, and the risk avoidance path for example, when a state variable of a lifestyle having a high prediction degree of a patient with hypertension among the plurality of lifestyles of the subject is generated, the risk path may provide the state variable to provide a simulation risk prediction value of a negative influence factor.

FIG. 5 is an exemplary view for explaining an example of evaluating a risk through a disease prevalence risk probability prediction and death risk of a statistical probability model generating unit 130 according to an exemplary embodiment of the present disclosure.

For example, referring to FIG. 5, the statistical probability model generating unit 130 may receive factors which are recognized by the individual as input 1. For example, the factor recognized by the individual may be a factor such as a lifestyle a body measurement value, and a disease history. The statistical probability model generating unit 130 may receive factors which are not recognized by the individual as input 2. The factors which are not recognized by the individual may be factors such as nutrition intake and clinical values

The statistical probability model generating unit 130 selects major state variables associated with a specific disease based on the input 1 and input 2 and predicts a currently possible disease probability of the subject. In the present disclosure, the prevalence probability of the metabolic disorders such as metabolic syndrome, obesity, hypertension, and diabetes may be predicted, The statistical probability model generating unit 130 may select one of risks represented as very high, high, normal, or low to provide the probability evaluation result. The disease risk predicting unit 140 may provide a personalized risk management information of the subject (individual) corresponding to each risk based on the probability evaluation result. The personalized risk management information of the subject (individual) may be a method which reduces the information such as hospital visit or health check-up and current possible disease probability, for a subject having a high probability.

The statistical probability model generating unit 130 may provide future disease incidence rusk evaluation of the metabolic disorder after a predetermined time interval has elapsed since the intermediate health condition is applied. The statistical probability model generating unit 130 classifies the risk evaluation result into the highest risk group, a high risk group, an intermediate risk group, and a low risk group to provide the risk evaluation result of the subject. The disease risk predicting unit 140 may provide personalized risk management information based on the risk evaluation result.

further, the statistical probability model generating unit 130 may provide the risk evaluation result of a future disease incidence risk and a death risk. For example, the final result may be a risk evaluation result of the chronic kidney disease or cardiovascular death winch may occur after the incidence of the metabolic disorder. The statistical probability model generating unit 130 classifies the risk evaluation for the final result into the highest risk group, a high risk group, an intermediate risk group, and a low risk group to provide the final result risk evaluation of the subject. The disease risk predicting unit 140 may provide personalized risk management information based on the final result risk evaluation result.

The disease risk predicting unit 140 may provide time series variation information of a negative influence factor of the metabolic disorders. Further, the disease risk predicting unit 140 may provide time series variation information of a positive influence factor. When the negative influence factor is virtually intervened, the disease risk predicting unit 140 may provide a positive time-series factor variation path. The disease risk predicting unit 140 may provide a virtual simulation risk prediction value before and after intervention.

According to an exemplary embodiment of the present disclosure, the individual's health state is improved based on the personalized risk management information provided by the disease risk predicting unit 140, a plurality of state variables, that is, factors which are recognized by the individual is input at every predetermined period (for example, one year), and the statistical probability model generating unit 130 may repeatedly predict an intermediate health condition, a result, and a final result based on the plurality of state variables.

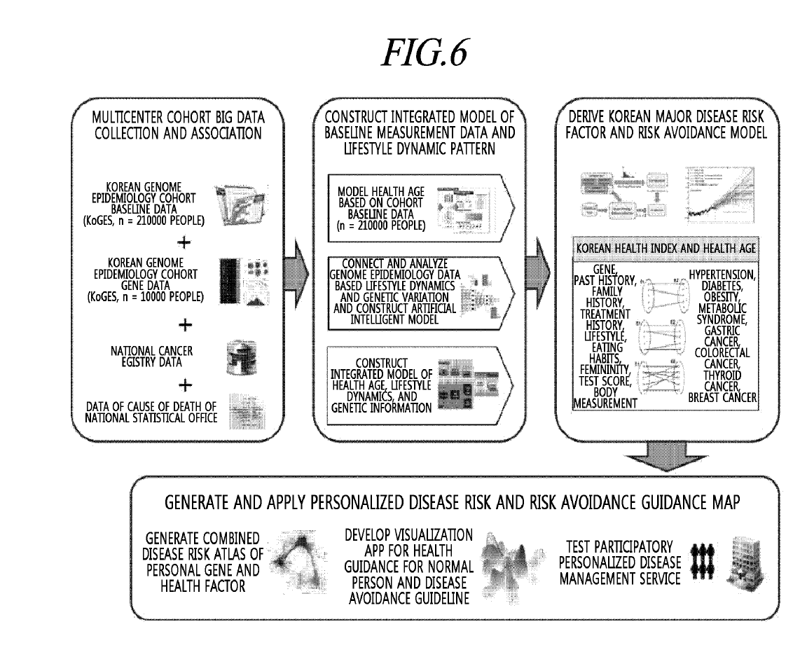

FIG. 6 is a vies for explaining an example of a process of predicting a metabolic disorder risk according to an exemplary embodiment of the present disclosure.

For example, referring to FIG. 6, the apparatus 100 for predicting a disease risk of metabolic disorders may be provided with multicenter cohort big data collection and association information from disease prediction server 200. The disease prediction server 200 may include Korean genome epidemiology cohort baseline data (KoGesmn=210000 people). Korean genome epidemiology cohort gene data (KoGES, n=10000 people). National Cancer Registry data, and data of cause of death of National Statistical Office, but is not limited thereto. For example, Korean genome epidemiology cohort baseline data (KoGesmn=210000 people), Korean genome epidemiology cohort gene data (KoGES, n=10000 people). National Cancer Registry data, and data of cause of death of National Statistical Office may be stored in the apparatus 100 for predicting a disease risk of metabolic disorders. p The apparatus 100 for predicting a disease risk of metabolic disorders may construct an integrated model of baseline measurement data and lifestyle dynamic patterns. The apparatus 100 for predicting a disease risk of metabolic disorders may model a health age based on the cohort baseline data (n=210000 people). The apparatus 100 for predicting a disease risk of metabolic disorders may connect and analyze the genome epidemiology data based lifestyle dynamics and genetic variation and construct an integrated model based on an artificial intelligence model. The apparatus 100 for predicting a disease risk of metabolic disorders may construct an integrated model of the health age, the lifestyle dynamics, and the genetic information.

Further, the apparatus 100 for predicting a disease risk of metabolic disorders may derive a Korean major disease risk factor and risk avoidance model. The apparatus 100 for predicting a disease risk of metabolic disorders may predict a disease such as hypertension, diabetes, obesity, metabolic syndrome, gastric cancer, colorectal cancer, thyroid cancer, or breast cancer through a machine learning model and a statistical model, based on input information such as genes, past history, family history, treatment history, lifestyle, eating habits, femininity, test scores, or body measurements.

The apparatus 100 for predicting a disease risk of metabolic disorders may generate a personalized disease risk and risk avoidance guidance map. The apparatus 100 for predicting a disease risk of metabolic disorders may improve the individual's health condition by providing the personalized disease risk and risk avoidance guidance map to reduce the disease risk probability.

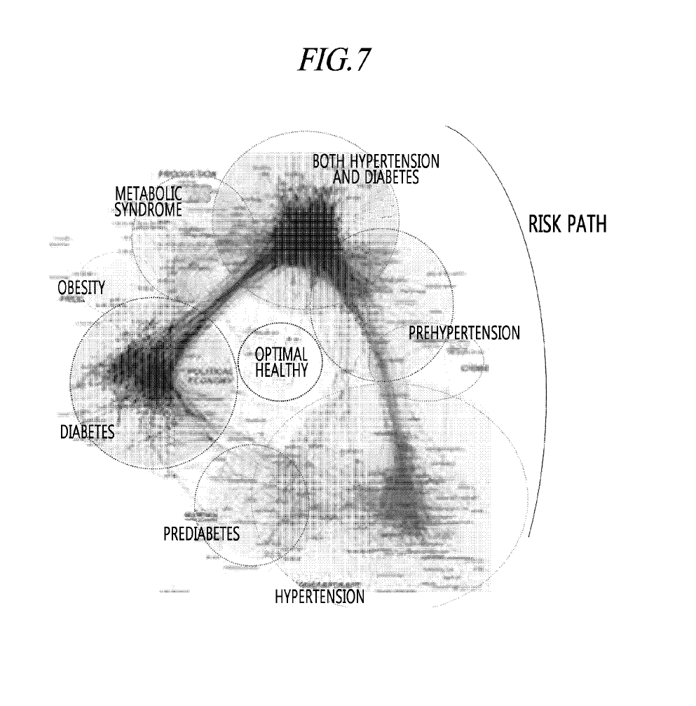

FIG. 7 a view illustrating clustering a plurality of metabolic disorders according to an exemplary embodiment of the present disclosure. Referring to FIG. 7, the machine learning model generating unit 120 may cluster a plurality of state variables corresponding to metabolic disorders.

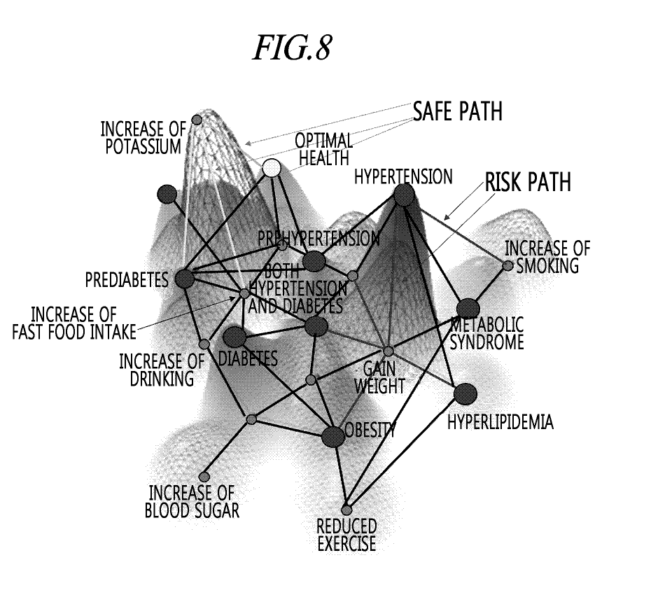

FIG. 8 is a view visualizing a guideline map for a disease risk of metabolic disorders according to an exemplary embodiment of the present disclosure. Referring to FIG. 3, the disease risk predicting unit 140 may visualize and provide a guidance map for a disease risk such as danger, safe, optimal, for the disease of metabolic disorders, based on the plurality of state variables.

Hereinafter, the result of the prehypertension and the hypertension incidence prediction among prediction results constructed by the statistical probability model generating 130 will be exemplified. For example, the statistical probability model generating unit 130 may evaluate con elation between the plurality of state variables (lifestyle and health condition variable) and the incidence of hypertension and clinical significance through a Cox proportional hazard model. Further, the statistical probability model generating unit 130 may construct a multivariate Cox proportional hazard model by including all variables having a significant correlation with the incidence of hypertension in a statistical model. The statistical probability model generating unit 130 selects variables having a significant eon el at ion with the incidence of disease in the multivariate Cox proportional hazard model and selects a final model based on the statistical explanatory power, clinical significance, and known epidemiological basis of the candidate variables obtained in this process.

The following Tables 1 to 3 are tables schematically representing variable selection results.

Table 1 represents a result of variables selected by applying a forward selection method among variable selection methods.

TABLE 1

| | Variables | P-value |
|---|---|---|
| 1 | Age | <.0001 |
| 2 | Education level | 0.0072 |
| 3 | Whether diabetes is affected | 0.2742 |
| 4 | Past history of hyperlipidemia | 0.0002 |
| 5 | Smoking status | 0.0022 |
| 6 | Amount of alcohol intakes | <.0001 |
| 7 | Body mass index | <.0001 |
| 8 | Liver function test (ALT) | <.0001 |
| 9 | Fasting blood sugar 100 mg/dL or | <.0001 |
| | higher | |
| 10 | Waist size male 90/female 85 or more | 0.0131 |
| 11 | Urine Dipstick test - protein detected | 0.0185 |
| 12 | Urine Dipstick test - sugar detected | 0.4736 |
| 13 | Family history of metabolic | 0.0186 |
| | cardiovascular disease | |
| 14 | Past history of heart failure | 0.0601 |
| 15 | Past history of coronary artery disease | 0.0212 |
| 16 | Past history of chronic pulmonary | <.0001 |
| | disease | |
| 17 | Past history of cerebrovascular disease | 0.0217 |

Table 2 represents variables selected by applying a backward elimination method among variable selection methods (backward: list of eliminated variables, SLS=0.05).

TABLE 2

| | Variables | P-value |
|---|---|---|
| 1 | Age | <.0001 |
| 2 | Education level | 0.0057 |
| 3 | Past history of hyperlipidemia | <.0001 |
| 4 | Smoking status | 0.0026 |
| 5 | Amount of alcohol intakes | <.0001 |
| 6 | Body mass index | <.0001 |
| 7 | Liver function test (ALT) | <.0001 |
| 8 | Fasting blood sugar 100 mg/dL or | <.0001 |
| | higher | |
| 9 | Waist size mail 90/female 85 or more | 0.0142 |
| 10 | Fasting blood sugar 125 mg/dL or | 0.0434 |
| | higher | |
| 11 | Urine Dipstick test - protein detected | <.0001 |
| 12 | Family history of metabolic | 0.0149 |
| | cardiovascular disease | |
| 13 | Past history of coronary artery disease | 0.0202 |
| 11 | Past history of chronic pulmonary | <.0001 |
| | disease | |
| 12 | Past history of cerebrovascular disease | 0.0254 |

Table 3 may represent variables selected by applying a stepwise selection method (stepwise: SLE=0.2, SLS=0.1) among variable selection methods.

TABLE 3

| | Variables | P-value |
|---|---|---|
| 1 | Age | 0.0033 |
| 2 | House income | 0.0029 |
| 3 | Amount of alcohol intakes | <.0001 |
| 4 | Body mass index | 0.004 |
| 5 | BUN | <.0001 |
| 6 | Liver function test (ALT) | 0.0095 |
| 7 | Hemoglobin | <.0001 |
| 8 | HbA1c | <.0001 |
| 9 | Fasting blood sugar 100 mg/dL or higher | <.0001 |
| 10 | Waist size mail 90/female 85 or more | <.0001 |
| 11 | Urine Dipstick test - myoglobin detected | 0.0003 |
| 12 | Iron intake amount | 0.0004 |
| 13 | Family history of metabolic cardiovascular | 0.0002 |
| | disease | |
| 14 | Past history of coronary artery disease | 0.0215 |
| 15 | Past history of chronic pulmonary disease | 0.0001 |

The statistical probability model generating unit 130 performs a process of combining two or more variables or simplifying an interval of variables to exclude multicollinearity and calculate a stable coefficient value for each variable (a plurality of state variables), during the process of selecting a final model based on candidate variables obtained through three steps of the variable selection method represented in Tables 1 to 3. For example, in the case of the urine dipstick test, the statistical probability model generating unit 130 combines glycosuria detection and protein in urine to be convened into a variable of urine score. Further, the age is classified into 40-49 years old/50-59 years old/over 60 years old, a continuous variable such as body measurements and clinical values are classified into a normal range and an out-of-normal risk level, or a normal range, a borderline level, and a risk level, based on the clinical criteria.

According to an exemplary embodiment of the present disclosure, influence of the risk facto of the metabolic disorder on the metabolic disorder is illustrated in a graph through the process of selecting a plurality of state variables by the statistical probability model generating unit 130.

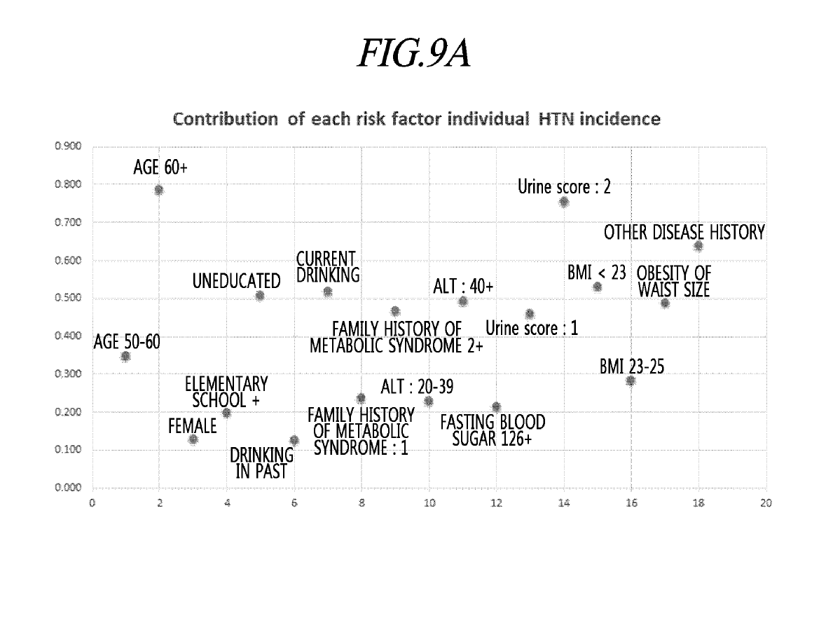

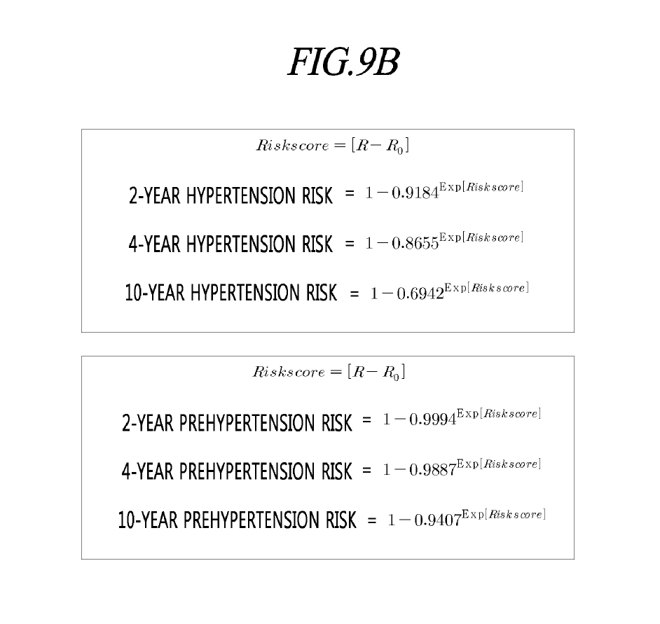

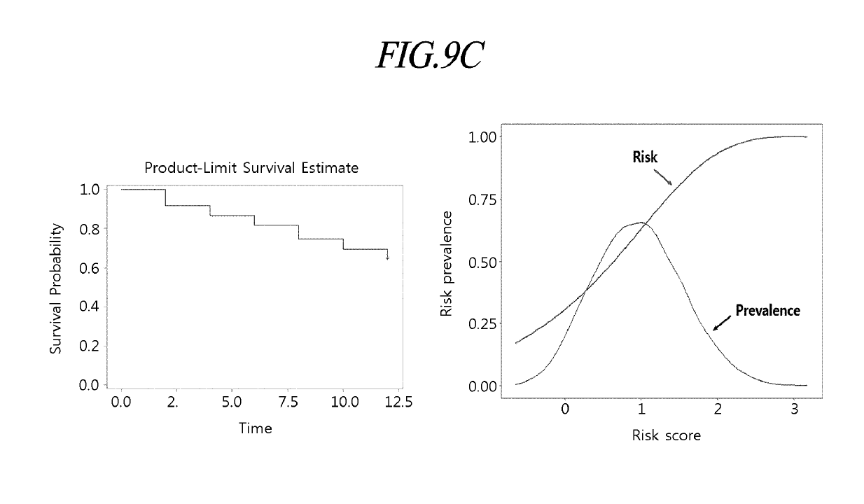

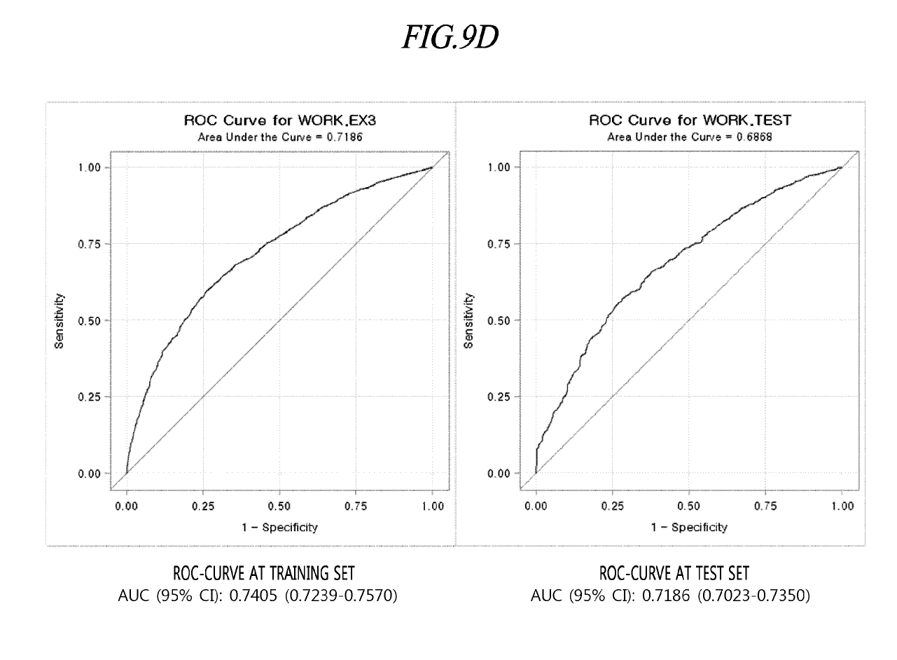

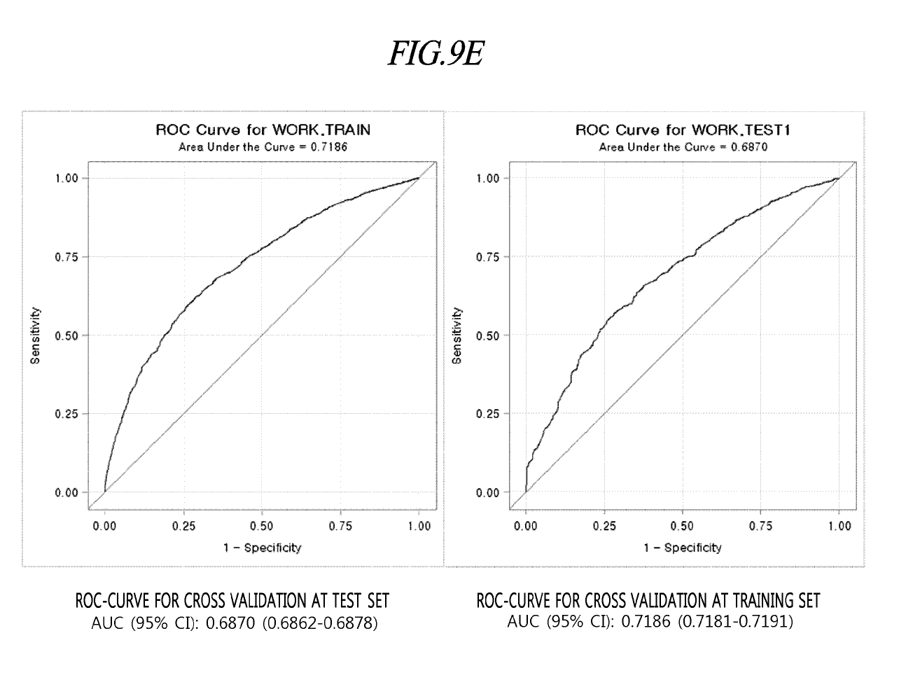

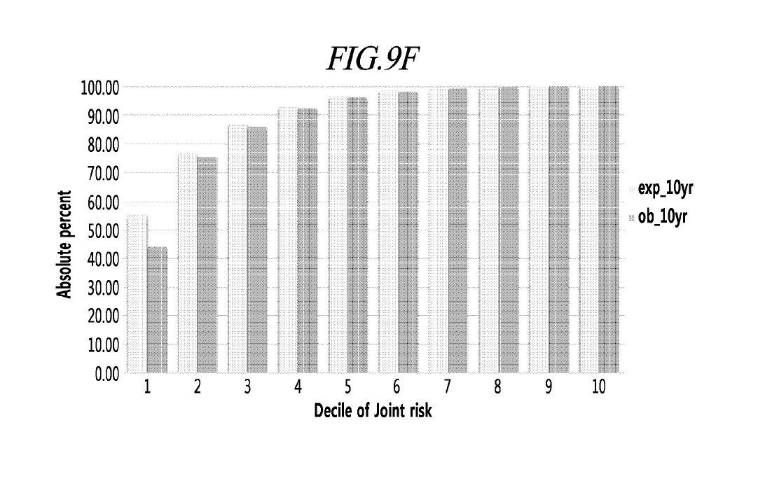

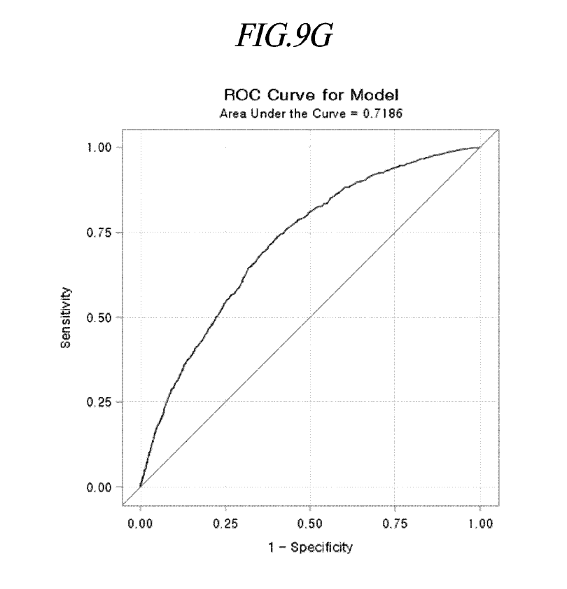

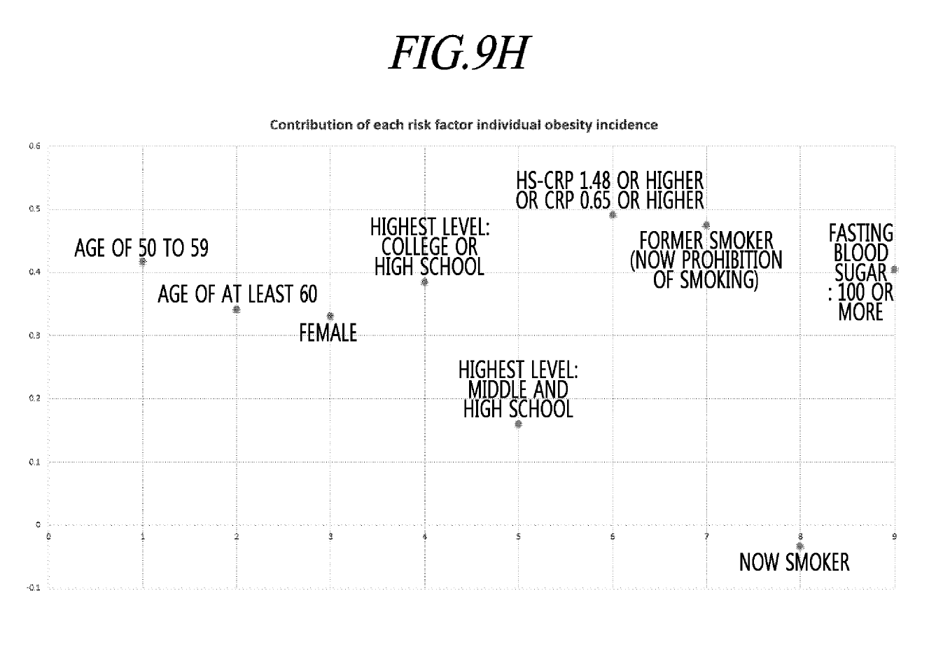

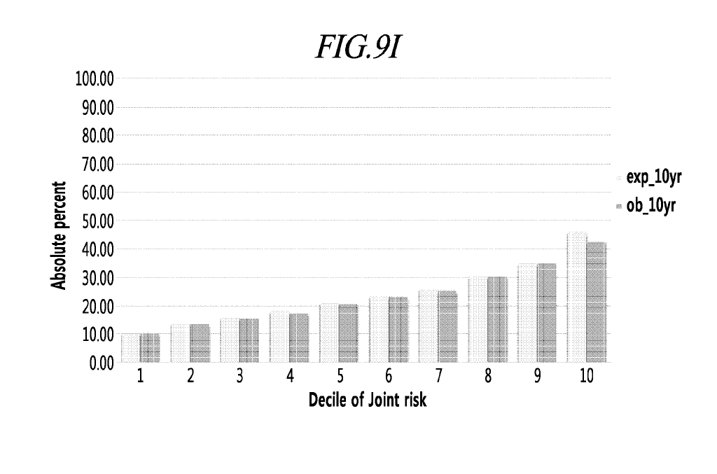

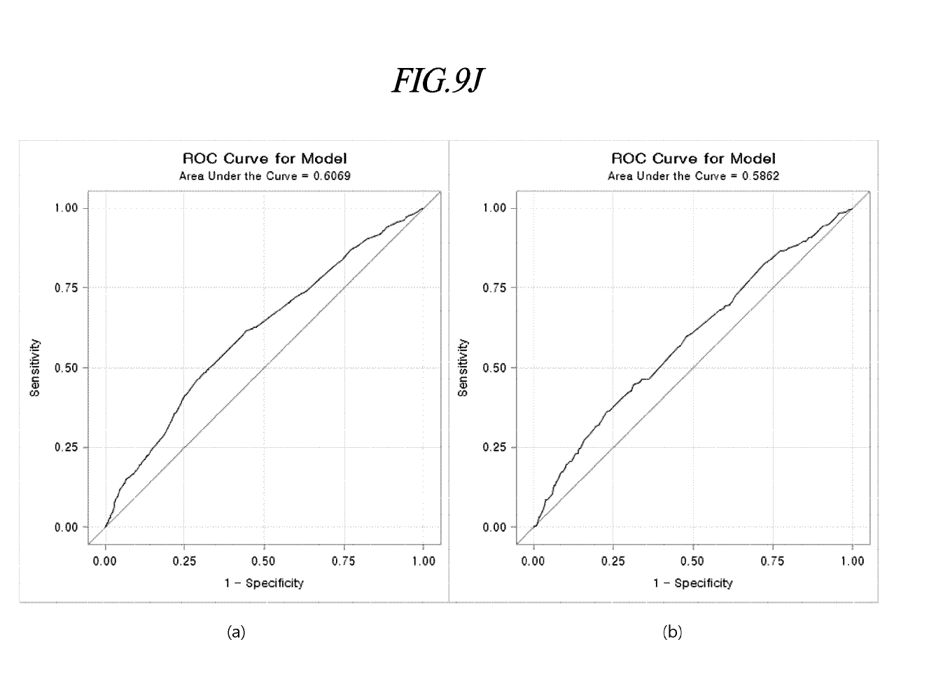

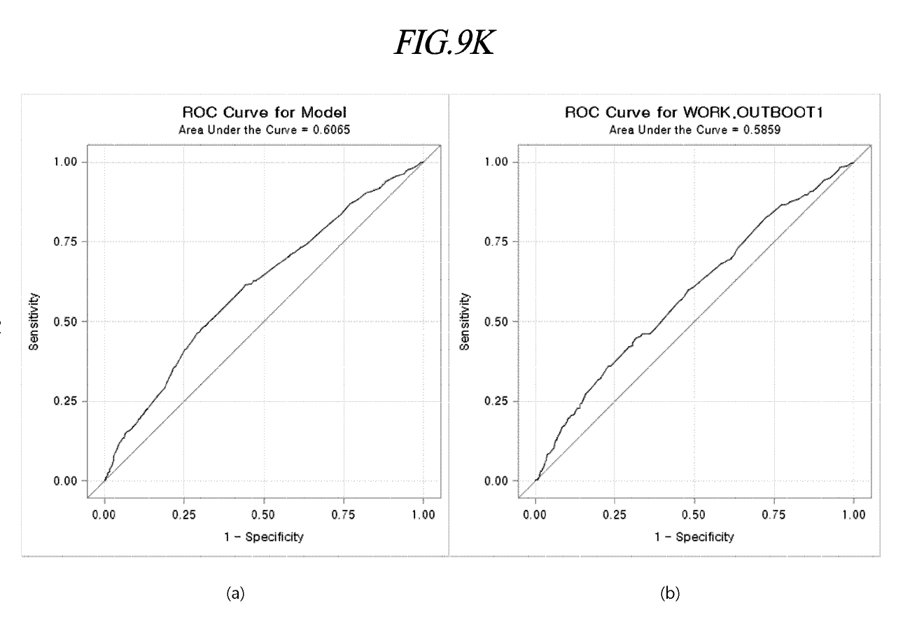

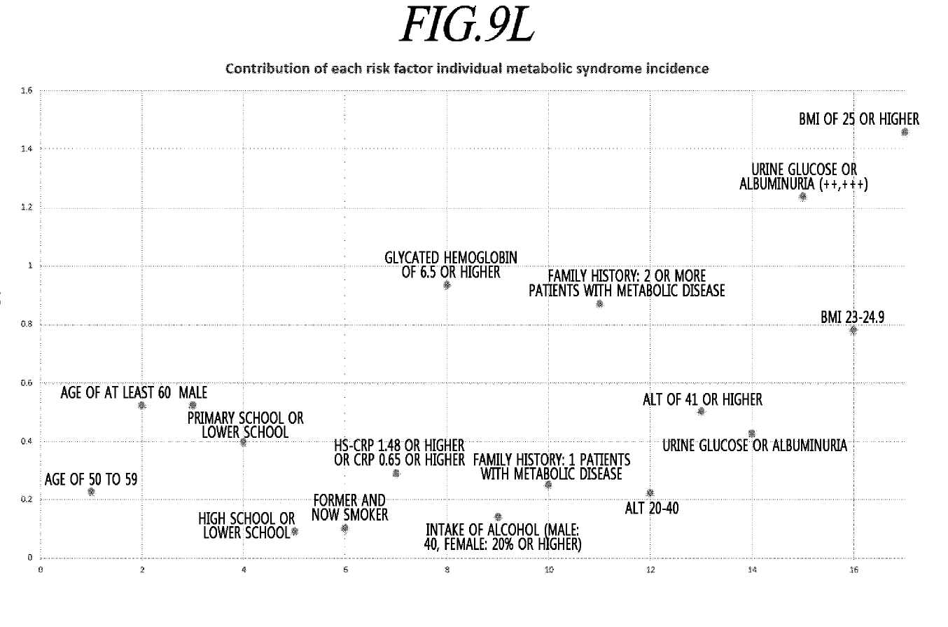

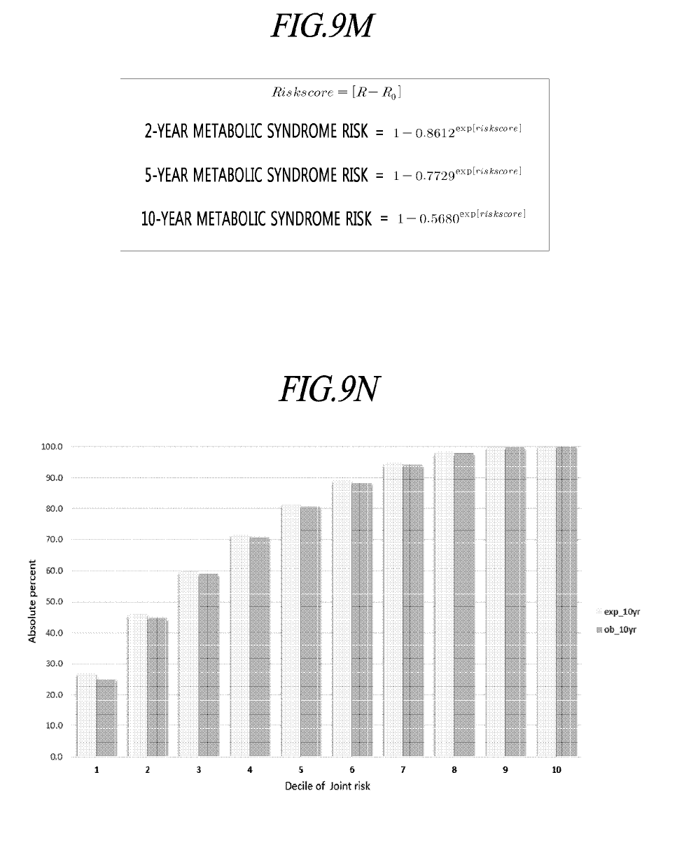

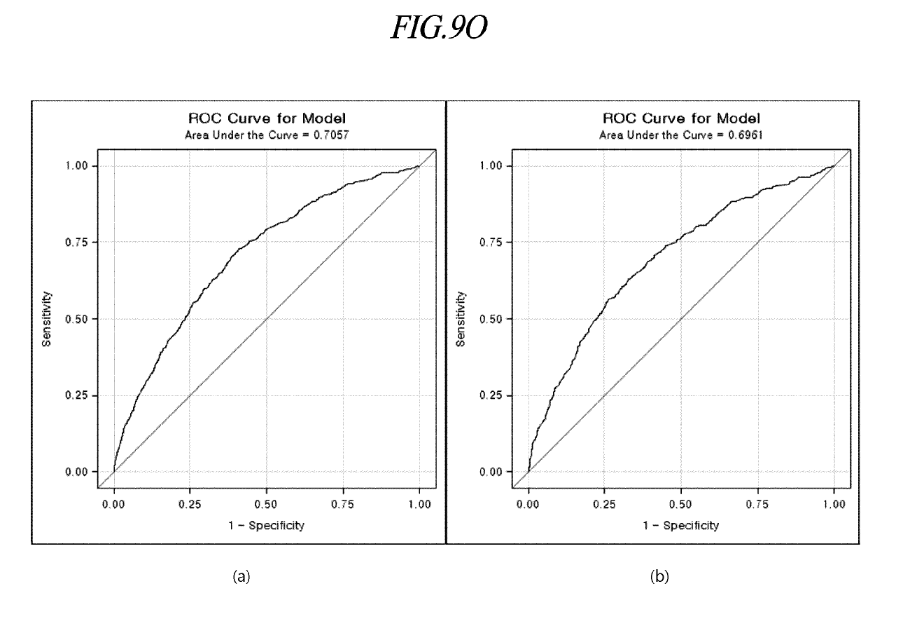

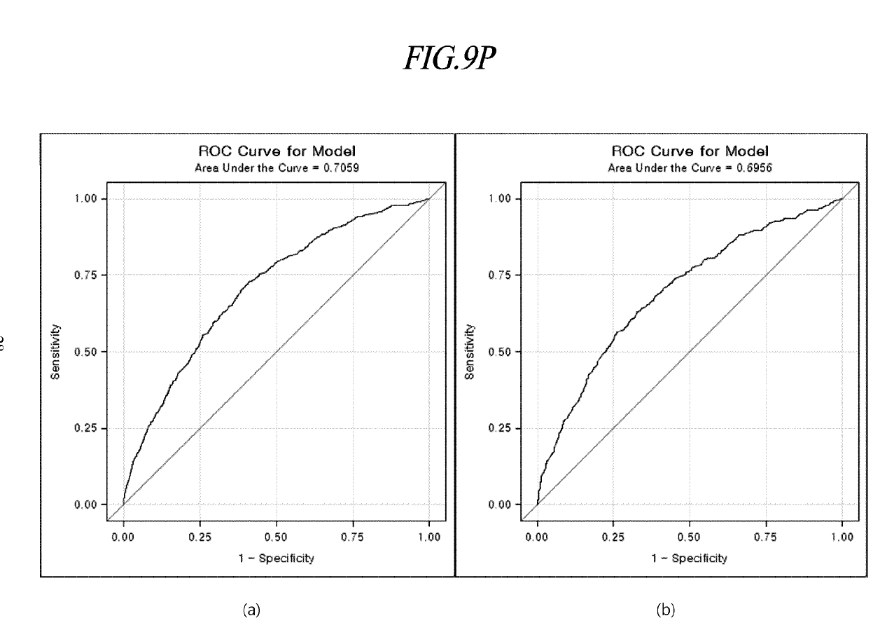

FIG. 9A is a view illustrating a graph of a correlation of a hypertension, risk factor Referring to FIG. 9A. the statistical probability model generating unit 130 calculates Joint Risk (JR) as represented in Equation 10 using an influence (b) value affecting on the disease incidence risk for every variable in the selected Cox proportional hazard model.

$${HR}_{i} = exp ( {β}_{i} ) \\ JR = exp ( {\sum }_{i} {β}_{i} ) \qquad [ Equation 10 ]$$

The statistical probability model generating unit 130 predicts an incidence risk R of a disease observed tor every subject and a risk Ro of a disease expected for every variable combination representing a baseline risk to finally calculate a unique risk score of each subject using the following Equation.

The incidence risk R of disease observed for every subject may be represented in Equation 11.

R=(β₁x₁+β₂x₂+ . . . +βₙxₙ) [Equation 11]

Further, the ask Ro of the disease expected for every variable combination representing a baseline risk may be represented in Equation 12.

R₀=(β₁x₁×c₁)+ . . . +(βₙxₙ×cₙ), (cₙ=frequency of exposure) [Equation 12]

The unique risk score of each subject may be represented in Equation 13.

Riskscore=R−R₀ [Equation 13]

A result of calculating a hypertension risk score using the above equation is as follows.

R(hypertension)=0.35081×[age 50-59 years old]+0.78914×[age: over 60 years old]+0.12973×[gender: female]+0.20087×[education level: elementary school or higher]+0.50856×[education level: uneducated]+0.12850×[drunk in the past & currently stop drinking]+0.51991×[currently drinker]+0.23994×[number of family history or metabolic cardiovascular disease: 1]+0.46804×[number of family history of metabolic cardiovascular disease: 2+]+0.23038×[ALT: 20-39]+0.49469×[ALT: 40+]+0.21599×[fasting blood sugar: 126+ +0.46171×[Urine score: 1]+0.75740×[Urine score: 2+]−0.53332×[body mass index: 23-25]−0.28629×[body mass index: 25+]+0.48784×[waist size or larger]+0.64224×[history of metabolic cardiovascular disease]

R(hypertension)=(0.31015*[gender=male]+0.64466*[education level=uneducated or elementary school]+0.30032*[education level=middle/high school]+0.25211*[urine dipstick test=1+]+0.67147*[urine dipstick test 2+ or above]+0.14519*[drinking status=currently normal drinker]+0.49028*[drinking status=excessive drinking (WHO basis)]+0.28945*[fasting blood sugar 100 mg/DL or higher]+0.20918*[ALT 20-39]+0.34625*[ALT 40+]+0.56323*[waste size (male 90 cm, female 85 or more)]

R0 (hypertension)=(0.35081×0.167937)+(0.78914×0.058857)+(0.12973×0.336888)+(0.20087×0.383394)+(0.50856×0.048626)+(0.12850×0.13931)+(0.51991×0.004758)+(0.23994×0.006942)+(0.4804×0.000212)+(0.23038×0.115931)+(0.49469×0.004099)+(0.21599×0.027350)+(0.46171×0.006736)+(0.75740×0.000024)+(−0.53332×0.147837)+(−0.28629×0.073394)+(0.4874×0.045542)+(0.6224×0.000048);

(prehypertension)=(0.31015*0.4359)+(0.64466*0.2029)+(0.30032*0.6239)+(0.25211*0.0713)+(0.67147*0.0032)+(0.14519*0.3935)+(0.49028*0.0628)+(0.28945*0.1631)+(0.20918*0.3499)+(0.34625*0.0610)+(0.56323*0.2012)

Referring to FIG. 9B, the disease risk predicting unit 140 calculates an incidence risk score of the hypertension and the pre-hypertension tor the entire subjects using the above equation and calculate 2-year, 4-year, and 10-year hypertension risk.

(a) of FIG. 9C is a graph of a hypertension incidence probability and (b) is a graph illustrating a risk score of a major factor of incidence of hypertension and 10-year hypertension risk.

According to an exemplary embodiment of the present disclosure, the statistical probability model generating unit 130 requires an incidence rate of each disease (hypertension, diabetes, obesity, metabolic syndrome, and chronical kidney disease), a mortality rate due to each disease, and modality data due to all cause of death in a normal population in order to complete a competitive risk model. The entire mortality data is calculated by the statistical data on the causes of death by age of the National Statistical Office and the mortality due to the obesity, the hypertension, and metabolic syndrome may be calculated using the risk in formation of the population contribution of deaths due to obesity, hypertension, and metabolic syndrome in the existing literature and statistical data on the causes of death by age of the National Statistical Office. The incidence rate by age for each disease may be calculated using health check-up sample cohort data of National health insurance service.

$${F}_{c} = \frac{exp ( {\sum }_{i ∈ c} {β}_{i} )}{{\prod }_{i ∈ c} prevalence ( i )} \\ {h}_{1} ( t ) = {\sum }_{c ∈ allpossiblecombinationsofriskfactors} \frac{1}{{F}_{c}} \\ r ( t ) = \frac{∈ cidencerate}{min ( ∈ cidencerate )} \\ {h}_{2} ( t ) = allcausemortalityrate - diseasespecificmortalityrate \\ {S}_{2} ( t ) = exp \{ - {\int }_{0}^{t} {h}_{2} ( u ) du \} \\ P \{ a , τ , r ( t ) \} = {\int }_{0}^{a + τ} {JR}^{*} {{h}_{1} ( t )}^{*} r ( t ) exp \{ - {\int }_{a}^{t} {JR}^{*} {{h}_{1} ( u )}^{*} r ( u ) du \} \{ {S}_{2} ( t ) / {S}_{2} ( a ) dt \} \qquad [ Equation 14 ]$$

The apparatus 100 for predicting a disease risk of metabolic disorders may construct a competitive risk model as represented in Equation 14, based on the calculated incidence rate of disease by age the mortality, and total mortalities. A validation process may be performed on the constructed competitive risk model by performing cross-validation by dividing the entire subjects into rives, tor the purpose of specificity(validity) validation.

Hereinafter, a predictive power validation process of a hypertension risk prediction model will be described. The predictive power and validation of the hypertension risk model may be performed using a total of three methods. Cross validation with internal specificity(validity) is performed using the ROC curve and an AUC value and an observed value and an incidence predicted value of hypertension incidence tor the calculated risk score value may be compared. A prediction degree of hypertension incidence prediction according to a risk score constructed by checking a sensitivity and a specificity(validity) by three methods of Youden index, a Distance to (0,1), and equality of sensitivity and specificity(validity) for an optimal cut-point of the hypertension risk may be evaluated.

Referring to FIG. 9D, an AUC value in a hypertension prediction model constructed using 70% training set (6657 subjects) is 0.7180 and a 95% confidence interval is 0.7023 to 0.7350. Further, an AUC value in a hypertension prediction model constructed using 30% training set (22853 subjects) is 0.7405 and a 95% confidence interval is 0.7239 to 0.7570.

The statistical probability model generating unit 130 may perform cross validation to verify the predictive power of the hypertension risk. According to the cross validation, permutation was performed on the training set and the test set 1000 times using a boot-strapping technique and 6657000 observed values from the training set and 2853000 observed values from the test set were confirmed as a result of permutation. Next, the cross validation was performed to identify whether the observed value and an expected value of the validation set match by applying the probability calculating method of the calculated model as it is. As a result, as illustrated in FIG. 9E, as a validation value of the predictive power of the hypertension risk for the training set, the AUC value was 0.7186 and 95% confidence interval was 0.7181 to 0.7191. Further, as illustrated in FIG. 9E, as the predictive power for the test set. the AUC value was 0.6870 and 95% confidence interval was 0.6862 to 0.6878.

FIG. 9F is a graph for comparing a hypertension incidence value and a prediction value for all subjects for 10 years. Referring, to the graph illustrated in FIG. 9F, the observed value and the predicted value of the hypertension incidence for the calculated risk core were compared (compared for incidence risk for 10 years). It is confirmed by this process that the actual hypertension incidence value for 10 years of monitoring period and the risk predicted through a model, were similar.

According to the exemplary embodiment of the present disclosure, the optimal cut-point, sensitivity, and specificity(validity) were confirmed using Youden index, Distance to (0,1) the principle of sensitivity and specificity equality for the training set.

As a result, the AUC value in the training set was calculated to be 0.7186 and the 95% confidence interval was 0.7023 to 0.7350. As a method for calculating Youden index, a maximum value (J=sensitivity+specificity−1) was used and the maximum value at this time was 0.3752. According to this, it was confirmed that the cut-point was 0.32488, the sensitivity was 0.73661, and the specificity was 0.59764. A minimum value calculated by the Distance to (0,1) method was 0.47389, the cut-point was 0.31509, the sensitivity was 0.69085, and the specificity was 0.64083. The sensitivity and specificity equality method refers to a case that the difference between the sensitivity and the specificity is minimum and according to this, it was confirmed that the minimum value calculated at this time was 0.00011, the cut-point was 0.31248, the sensitivity was 0.66183, and the specificity was 0.66172. FIG. 9G is a graph illustrating a predictive power (AUC is 0.7186) of the hypertension prediction model using the training set.

Table 4 may be a result of an optimal cut-point, a sensitivity, and a specificity(validity) using the above-described three methods.

TABLE 4

| | cut-point | Sensitivity | Specificity |
|---|---|---|---|
| Yoden index | 0.32488 | 0.73661 | 0.59764 |
| Distance to (0, 1) | 0.31509 | 0.69085 | 0.64083 |
| Sensitivity, Specificity | 0.31248 | 0.66183 | 0.66172 |
| equality | | | |

A prediction result for the diabetes (2) among prediction results constructed by the above-described statistical probability model generating unit 130 is as follows. The statistical probability model generating unit 130 divided community cohort data of Disease management center into 80% training set and 20% test set with respect to the subjects and constructed the following model with the training set. The statistical probability model generating unit 130 applied variables which are significant in the diabetes prevalence risk prediction model to a univariate Cox proportional hazard model including the age of the subject as a default variable to evaluate the correlation and select candidate variables.

However, in this case, a variable which may be changed at every measurement in the repeated measurement data among the variables of the community cohort data was changed into a time-dependent type to be applied to the multinomial Cox regression analysis. As the variables with fixed values such as an age of first menstrual period or an education level, variables which were time-independent and initially measured were applied. In the following tables, the above-described process and the candidate variables selected by the process were represented according to the gender in a descending order of Harrel's C concordance index.

Table 5 represents candidate variables of a prediction model of a diabetes risk for a male subject.

TABLE 5

| | | | P- | Harrell's |
|---|---|---|---|---|
| Name of variable | HR (95% CI) | | value | C |
| Ratio of waist to hip | 3.35 | (1.71-6.57) | 0.0004 | 0.674 |
| circumference | 6.55 | (3.4-12.63) | <.0001 | |
| | 11.19 | (5.77-21.69) | <.0001 | |
| γ-GTP | 2.32 | (1.03-5.22) | 0.042 | 0.673 |
| | 3.72 | (1.71-8.09) | 0.0009 | |
| | 7.71 | (3.59-16.55) | <.0001 | |
| Triglyceride | 1.7 | (1.07-2.72) | 0.0258 | 0.65 |
| | 2.43 | (1.55-3.79) | 0.0001 | |
| | 4.03 | (2.63-6.17) | <.0001 | |
| ALT liver level | 1.47 | (1.01-2.15) | 0.0451 | 0.649 |
| | 3.93 | (2.63-5.88) | <.0001 | |
| BMI: body mass index | 1.36 | (0.91-2.02) | 0.1341 | 0.638 |
| | 2.06 | (1.42-2.99) | 0.0001 | |
| | 3.38 | (2.35-4.86) | <.0001 | |
| DBP: diastolic blood pressure | 1.48 | (0.95-2.3) | 0.082 | 0.617 |
| | 2.43 | (1.58-3.73) | <.0001 | |
| | 2.92 | (1.93-4.44) | <.0001 | |
| SBP: systolic blood pressure | 1.38 | (0.92-2.07) | 0.1206 | 0.606 |
| | 2.09 | (1.44-3.05) | 0.0001 | |
| | 2.29 | (1.53-3.44) | 0.0001 | |
| Past history of hypertension - | 2.34 | (1.7-3.21) | <.0001 | 0.595 |
| whether to be diagnosed | | | | |
| Smoking pack-year | 1.27 | (0.84-1.91) | 0.2528 | 0.592 |
| | 2.06 | (1.41-3.02) | 0.0002 | |
| | 1.93 | (1.23-3.04) | 0.0043 | |
| HDL-cholesterol | 0.69 | (0.51-0.93) | 0.0154 | 0.59 |
| | 0.54 | (0.37-0.8) | 0.0022 | |
| | 0.58 | (0.4-0.84) | 0.0036 | |
| Income | 0.88 | (0.63-1.23) | 0.4421 | 0.577 |
| | 0.65 | (0.45-0.94) | 0.0229 | |
| | 0.6 | (0.34-1.06) | 0.0801 | |
| Fiber(g) | 1.07 | (0.73-1.55) | 0.7386 | 0.575 |
| | 1.07 | (0.74-1.56) | 0.7048 | |
| | 1.48 | (1.03-2.13) | 0.0346 | |
| Hemoglobin | 1.52 | (0.74-3.13) | 0.2512 | 0.575 |
| | 2.26 | (1.09-4.66) | 0.0281 | |
| Past history of chronic gastri- | 0.58 | (0.41-0.82) | 0.0022 | 0.575 |
| tis - whether to be diagnosed | | | | |
| Past history of hyperlipid- | 2.43 | (1.42-4.19) | 0.0013 | 0.573 |
| emia - whether to be | | | | |
| diagnosed | | | | |
| Uric Acid | 1.32 | (0.66-2.64) | 0.4244 | 0.571 |
| | 1.72 | (0.9-3.31) | 0.1032 | |
| | 1.87 | (0.98-3.57) | 0.0565 | |
| Family history of hyper- | 1.32 | (0.97-1.79) | 0.0753 | 0.568 |
| tension - family history | | | | |
| or not | | | | |
| Vitamin C[Vit. C(mg)] | 0.98 | (0.68-1.41) | 0.9061 | 0.567 |
| | 1.06 | (0.74-1.52) | 0.759 | |
| | 1.36 | (0.95-1.95) | 0.0931 | |
| Total cholesterol | 1.22 | (0.85-1.77) | 0.2776 | 0.567 |
| | 1.3 | (0.89-1.9) | 0.1739 | |
| | 1.56 | (1.08-2.25) | 0.0182 | |
| Ash (mg) | 1.21 | (0.81-1.8) | 0.3516 | 0.567 |
| | 1.27 | (0.86-1.88) | 0.2213 | |
| | 1.44 | (0.98-2.13) | 0.0625 | |
| Potassium [K(mg)] | 1.12 | (0.76-1.64) | 0.5693 | 0.566 |
| | 1.24 | (0.85-1.8) | 0.2704 | |
| | 1.39 | (0.95-2.03) | 0.0867 | |
| Past history of allergic | 0.59 | (0.28-1.25) | 0.1704 | 0.563 |
| disease - whether to be diagnosed | | | | |
| Retinol (ug) | 0.7 | (0.48-1.02) | 0.0623 | 0.563 |
| | 0.82 | (0.57-1.17) | 0.2662 | |
| | 0.88 | (0.62-1.25) | 0.4802 | |
| Marriage state | 0.65 | (0.38-1.09) | 0.1013 | 0.563 |
| Family history of diabetes - | 1.68 | (1.14-2.47) | 0.0082 | 0.562 |
| family history or not | | | | |
| Family history of heart | 0.51 | (0.23-1.14) | 0.101 | 0.561 |
| disease - family history or not | | | | |
| Occupation | 0.63 | (0.42-0.94) | 0.022 | 0.561 |
| | 0 | (—) | 0.9934 | |
| | 0.93 | (0.68-1.26) | 0.6272 | |
| Sodium [Na(mg)] | 1.35 | (0.9-2.02) | 0.1485 | 0.561 |
| | 1.58 | (1.06-2.35) | 0.0249 | |
| | 1.33 | (0.89-2) | 0.1609 | |
| Education level | 1.08 | (0.77-1.52) | 0.6519 | 0.56 |
| | 0.68 | (0.43-1.07) | 0.0954 | |
| Past history of angina | 2.43 | (0.89-6.58) | 0.0818 | 0.56 |
| pectoris/myocardial infarc- | | | | |
| tion - whether to be diagnosed | | | | |
| Thyroid gland disease - | 2.28 | (1.01-5.12) | 0.0468 | 0.557 |
| whether to be diagnosed | | | | |

Table 6 represents candidate variables of a prediction model of a diabetes risk a female subject.

TABLE 6

| | | | P- | Harrell’s |
|---|---|---|---|---|
| Name of variable | HR (95% CI) | | value | C |
| Triglyceride | 2.71 | (1.8-4.09) | <.0001 | 0.718 |
| | 4.15 | (2.78-6.19) | <.0001 | |
| | 6.55 | (4.41-9.74) | <.0001 | |
| BMI: body mass index | 2.34 | (1.48-3.69) | <.0001 | 0.713 |
| | 3.12 | (2-4.86) | <.0001 | |
| | 6.3 | (4.15-9.56) | <.0001 | |
| γ-GTP | 2.18 | (1.64-2.9) | <.0001 | 0.702 |
| | 3.79 | (2.78-5.17) | <.0001 | |
| | 4.51 | (3.1-6.55) | <.0001 | |
| DBP: diastolic blood pressure | 2.13 | (1.51-3.01) | <.0001 | 0.688 |
| | 2.33 | (1.61-3.38) | <.0001 | |
| | 3.82 | (2.7-5.39) | <.0001 | |
| SBP: systolic blood pressure | 1.67 | (1.17-2.39) | 0.0048 | 0.684 |
| | 1.95 | (1.36-2.79) | 0.0003 | |
| | 3.36 | (2.38-4.75) | <.0001 | |
| Ratio of waist to hip | 2 | (1.39-2.88) | 0.0002 | 0.679 |
| circumference | 2.48 | (1.73-3.55) | <.0001 | |
| | 3.22 | (2.28-4.53) | <.0001 | |
| Past history of hypertension- | 2.54 | (1.96-3.3) | <.0001 | 0.678 |
| whether to be diagnosed | | | | |
| HDL-cholesterol | 0.69 | (0.52-0.91) | 0.0078 | 0.672 |
| | 0.53 | (0.38-0.73) | 0.0001 | |
| | 0.38 | (0.27-0.53) | <.0001 | |
| Total cholesterol | 2.03 | (1.43-2.89) | 0.0001 | 0.665 |
| | 1.91 | (1.33-2.72) | 0.0004 | |
| | 2.23 | (1.56-3.18) | <.0001 | |
| ALT liver function level | 1.73 | (1.36-2.2) | <.0001 | 0.664 |
| | 3.68 | (2.53-5.34) | <.0001 | |
| Hemoglobin | 1.64 | (1.21-2.23) | 0.0015 | 0.65 |
| | 2.7 | (1.72-4.26) | <.0001 | |
| Family history of diabetes - | 2.08 | (1.54-2.8) | <.0001 | 0.649 |
| whether to have family history | | | | |
| Past history of angina pec- | 3.5 | (1.65-7.44) | 0.0011 | 0.648 |
| toris/myocardial infarction - | | | | |
| whether to be diagnosed | | | | |
| Past history of chronic gastri- | 0.78 | (0.59-1.03) | 0.0806 | 0.646 |
| tis - whether to be diagnosed | | | | |
| Family history of hyper- | 1.25 | (0.95-1.63) | 0.106 | 0.645 |
| tension - whether to have | | | | |
| family history | | | | |
| Albumin | 1.35 | (1.03-1.77) | 0.027 | 0.645 |
| | 1.36 | (0.97-1.9) | 0.0766 | |
| | 1.23 | (0.84-1.8) | 0.2902 | |
| Potassium [Ca(mg)] | 0.72 | (0.52-0.99) | 0.0423 | 0.644 |
| | 0.9 | (0.66-1.22) | 0.4976 | |
| | 0.85 | (0.62-1.15) | 0.2919 | |
| Occupation | 0.57 | (0.25-1.31) | 0.1848 | 0.644 |
| | 1.1 | (0.87-1.39) | 0.4395 | |
| | 1 | (0.62-1.64) | 0.9861 | |
| Income | 0.77 | (0.57-1.04) | 0.0855 | 0.644 |
| | 0.85 | (0.61-1.18) | 0.3226 | |
| | 0.68 | (0.38-1.23) | 0.2042 | |
| Education level | 0.86 | (0.65-1.14) | 0.2948 | 0.644 |
| | 0.5 | (0.26-0.97) | 0.0397 | |
| Smoking pack-year | 1.67 | (0.98-2.86) | 0.0612 | 0.643 |
| | 0 | (—) | 0.992 | |
| | 4.86 | (0.68-34.69) | 0.1151 | |
| Fat | 0.89 | (0.67-1.19) | 0.4369 | 0.642 |
| | 0.99 | (0.73-1.34) | 0.9343 | |
| | 0.67 | (0.46-0.99) | 0.0422 | |
| Daily alcohol intakes | 1.1 | (0.81-1.49) | 0.5598 | 0.642 |
| | 0.88 | (0.51-1.52) | 0.6556 | |
| | 1.31 | (0.65-2.66) | 0.4526 | |
| | 2.63 | (0.84-8.26) | 0.0974 | |
| Age of first birth | 1 | (0.73-1.35) | 0.9797 | 0.641 |
| | 0.91 | (0.67-1.23) | 0.5482 | |
| | 0.77 | (0.52-1.14) | 0.1925 | |
| [Retinol(ug)] | 0.79 | (0.58-1.08) | 0.1382 | 0.64 |
| | 0.75 | (0.55-1.04) | 0.0871 | |
| | 0.83 | (0.6-1.14) | 0.2477 | |

The following Equation describes a process of constructing a final prediction model based on the above candidate variables (sea Tables 4 and 5). During the process of constructing the final prediction model, male and female subjects are divided to apply the forward selection method, the backward elimination method, the stepwise method, and the selection method to perform a secondary variable process and the existing literature is reviewed to select clinically significant variables as final variables. Based on this, the final diabetes prediction models for the male subject and the female subject were constructed as follows.

R(female)=0.00995*[age]+0.03922*[pulse rate=2Q]+0.02956*[pulse rate=3Q]+0.29267*[pulse rate=4Q]+0.40547*[body mass index=2Q]+0.50078*[body mass index=3Q]+0.59333*[body mass index=4Q]+0.22314*[systolic blood pressure=2Q]+0.45747*[systolic blood pressure=3Q]+0.41211*[systolic blood pressure=4Q]+0.17395*[waist to hip circumference ratio=2Q]+0.36464*[waist to hip circumference ratio=3Q]+0.51282*[waist to hip circumference ratio=4Q]+0.07696*[γ-GTP=2Q]+0.31481*[γ-GTP=3Q]+0.30010*[γ-GTP=4Q]+0.29267*[total cholesterol=2Q]+0.19062*[total cholesterol=3Q]+0.26236*[total cholesterol=4Q]+0.43178*[whether to take hysterectomy=yes]+0.14842*[ALT liver level=slightly increased]+0.37844*[ALT liver level=moderately increased]

R(male)=0.12222*[γ-GRP=2Q]+0.27003*[γ-GTP=3Q]+0.58779*[γ-GTP=4Q]+0.02956*[waist to hip circumference ratio=2Q]+0.23111*[waist to hip circumference ratio=3Q]+0.54232*[waist to hip circumference ratio=4Q]+0.23111*[ALT=slightly increased]+0.47000*[ALT=moderately increased]+0.23902*[family history of diabetes=yes]+0.21511*[systolic blood pressure=3Q]+0.32208*[systolic blood pressure=4Q]−0.09431*[HDL=2Q]−0.15082*[HDL=3Q]−0.11653*[HDL=4Q]+0.15700*[drinking state=upper 50%]

The statistical probability model generating unit 130 calculated a risk score of each subject of 20% test set using result parameter values of prediabetes prediction models for male and female subjects constructed using the 80% training set. The predictive power of the model was verified through Harrell's C concordance index which compares the risk score and a time-until-event until actual prediabetes occurs. In the case of the prediabetes prediction model of the male subject, the predictive power of the training set was 0.6327 and the predictive power verified in the test set was 0.6137. In the case of the prediabetes prediction model of the female subject, the predictive power of the training set was 0.6968 and the predictive power verified in the test set was 0.6633.

For the prediction model of obesity among the prediction results constructed by the statistical probability model generating unit 130, the actual age group of the community cohort which was an actual data source was middle and prune ages of 40 to 70 years old and weigh change due to obesity was not observed at a level required for the study, so that only the overweight (2) was analyzed. The prediction result for overweight is illustrated in the graph of FIG. 9H. First, the correlation between the lifestyle, the health condition variable, and the incidence of overweight and the clinical Significance are evaluated by the Cos proportional hazard model and all variables having the significant correlation with the incidence of the overweight are included in the model to construct the multivariate Cox proportional hazard model. Variables having the significant correlation with the incidence of each disease in the multivariate Cox proportional hazard model are selected and a final model is selected based on the statistical explanatory power, clinical significance, and known epidemiological basis of the candidate variables obtained in this process. FIG. 9H is a view illustrating the correlation between the overweight incidence and the risk factor.

The process of calculating a join risk JR using a b value in the selected Cox proportional hazard model and calculating a unique risk score of each subject have the same equation and process as the hypertension prediction model described above. A result of calculating an overweight risk score is as follows.

R=(0.48390453*[40-49 years old]+0.41059621*[50-59 years old]+0.31819286*[sex=female]+0.378146797*[education=college or above]+0.137845916*[education=middle or high]+0.454680575*b_SL_CRP1+0.544133653*[past smoker]+0.057786443*[current smoker]+0.483874227*[fasting glucose≥100];

### R₀1.20881

A risk score of metabolic syndrome incidence for all subjects was calculated using the above equation and 2-year, 4-year, and 10-year overweight risks were calculated based thereon.

FIG. 9I is a bar graph for comparing an overweight risk score for 10 years and an incidence probability observed in actual study subjects according to the decile interval of the risk score.

The method of completing a competitive risk model has the same process, equation, and data source as those of the hypertension incidence model described above so that the description will be omitted. The validation is performed on the competitive risk model constructed based on the calculated incidence rate of disease by age, the mortality, and total mortalities by performing cross-validation by dividing the entire subjects into fives.

The predictive power validation process of a prediction model of an overweight risk will be described with reference to FIG. 9J. (a) of FIG. 9J is a predictive power of an overweight prediction model using repeatedly measured data of a training set (3089 subjects) and (b) is a predictive power of an overweight prediction model using repeatedly measured data of a test set (1324 subjects). The predictive power and validation of the overweight risk model may be performed using a total of three methods. Cross validation with internal specificity(validity) is performed using the ROC curve and an AUC value and an observed value and an incidence predicted value of overweight incidence for the calculated risk score value may be compared. A prediction degree of overweight incidence prediction according to a risk score constructed by checking a sensitivity and a specificity(validity) by three methods of Youden index, a Distance to (0,1), and equality of sensitivity and specificity(validity) for an optimal cut-point of the hypertension risk is evaluated. In the graph illustrated in FIG. 9J, an AUC value in the overweight prediction model constructed using 70% training set (3089 subjects) was calculated to be 0.6069 and a confidence interval was 0.5840 to 0.6298. An AUC value in the overweight prediction model constructed using 30% testing set (1324 subjects) was calculated to be 0.5862 and the 95% confidence interval was 0.5509 to 0.6215.

The statistical probability model generating unit 130 may perform cross validation to verify the predictive power of the overweight risk. According to the cross validation, similarly to the above-described hypertension incidence model, permutation was performed on the training set and the test set 1000 times using a boot-snapping technique and 16469000 observed values from the training set and 6962000 observed values from the test set were confirmed as a result of permutation. Next, the cross validation was performed to identify whether the observed value and an expected value of the validation set match by applying the probability calculating method of the calculated model as it is. As a result, as illustrated in the following drawing, as the validation value of the predictive power of the hypertension risk for the training set, the AUC is 0.6065 and the 95% confidence interval is 0.6058 to 0.6073. Further, as illustrated in the right drawing, as the predictive power for the test set, the AUC is 0.5859 and the 95% confidence interval is 0.5848 to 0.5870.

The statistical probability model generating unit 130 confirms the optimal cut-point, sensitivity, and specificity(validity) using Youden index, Distance to (0,1), the principle of sensitivity and specificity equality for the training set. In order to calculate the Youden index, a maximum value (J=sensitivity+specificity−1) is used and thus it was confirmed that the cut point was 0.34444, the sensitivity was 0.61777, and the specificity was 0.69643. A minimum value D calculated by the Distance to (0,1) method was 0.58615, the cut-point was 0.35306, the sensitivity was 0.61777, and the specificity was 0.69643. The sensitivity and specificity equality method means that the difference between the sensitivity and the specificity is minimum and thus the cut-point was 0.35304, the sensitivity was 0.56752, and the specificity was 0.60386.

Table 7 is a result of confirming an optimal cut-point, a sensitivity, and a specificity(validity) of an overweight risk using three methods.

TABLE 7

| | cut-point | Sensitivity | Specificity |
|---|---|---|---|
| Yoden index | 0.34444 | 0.71195 | 0.46216 |
| Distance to (0, 1) | 0.35396 | 0.61777 | 0.69643 |
| Sensitivity, Specificity | 0.35304 | 0.56752 | 0.60386 |
| equality | | | |

According to an exemplar v embodiment of the pit sent disclosure a process and a result of constructing a prediction model for metabolic syndrome (4) among prediction results constructed by the statistical probability model generating unit 130 is as follows. First, the correlation between the lifestyle, the health condition variable, and the incidence of metabolic syndrome and the clinical significance are evaluated by the Cox proportional hazard model and all variables having the significant correlation with the metabolic syndrome are included in the model to construct the multivariate Cox proportional hazard model. Variables having the significant correlation with the incidence of each disease in the multivariate Cox proportional hazard model are selected and a final model is selected based on the statistical explanatory power, clinical significance, and known epidemiological basis of the candidate variables obtained in this process. FIG. 9I is a graph illustrating a correlation between incidence of the metabolic syndrome and risk factors.

The process of calculating a join risk JR using a b value in the selected Cox proportional hazard model and calculating a unique risk score of each subject have the same equation and process as the hypertension prediction model described above. The result of calculating a metabolic syndrome risk score is as follows.

R=(0.19128*[age=50-59]+0.49768*[age=60-69]+0.51076*[gender=male+0.04479*[education level=middle/high school]+0.40455*[education level=elementary school or uneducated]+0.09120*[smoking=currently stop smoking or smoking]+0.27919*[CRP=abnormal]+0.93949*[glycated hemoglobin=abnormal]+0.15759*[drinking =WHO standard or higher]+0.29207*[number of family history of metabolic cardiovascular disease=1]+0.69454*[number of family history of metabolic cardiovascular disease=2+]+0.26725*[ALT=20-39]+0.55180*[ALT=40+]+0.45048*[urine dipstick=1+]+1.27320*[urine dipstick=2+]+0.81051*[body mass index=23-24.9]+1.47086*[body mass index=25+];

In this case, R₀=2.07417.

As illustrated in FIG. 9M, a metabolic syndrome risk score for all subjects was calculated using the above equation and 2-year, 4-year, and 10-year metabolic syndrome risks were calculated based thereon.

In order to complete a competitive risk model, an incidence rate of metabolic syndrome, a mortality rate due to each disease, and mortality data due to all cause of death in a normal population are required. The entire mortality data is calculated by the statistical data on the causes of death by age of the National Statistical Office and the mortality due to the obesity, the hypertension, and metabolic syndrome may be calculated using the risk information of the population contribution of deaths due to metabolic syndrome in the existing literature and statistical data on the causes of death by age of the National Statistical Office. The incidence rate by age for each disease may be calculated using, health check-up sample cohort data of National health insurance service.

$${F}_{c} = \frac{exp ( {\sum }_{i ∈ c} {β}_{i} )}{{\prod }_{i ∈ c} prevalence ( i )} \\ {h}_{1} ( t ) = {\sum }_{c ∈ allpossiblecombinationsofriskfactors} \frac{1}{{F}_{c}} \\ r ( t ) = \frac{∈ cidencerate}{min ( ∈ cidencerate )} \\ {h}_{2} ( t ) = allcausemortalityrate - diseasespecificmortalityrate \\ {S}_{2} ( t ) = exp \{ - {\int }_{0}^{t} {h}_{2} ( u ) du \} \\ P \{ a , τ , r ( t ) \} = {\int }_{a}^{a + τ} {JR}^{*} {{h}_{1} ( t )}^{*} r ( t ) exp \{ - {\int }_{a}^{t} {JR}^{*} {{h}_{1} ( u )}^{*} r ( u ) du \} \{ {S}_{2} ( t ) / {S}_{2} ( a ) dt \} \qquad [ Equation 14 ]$$

The statistical probability model generating unit 130 constructs a competitive risk model as represented in above Equation, based on the calculated incidence rate of disease by age, the mortality, and the total mortalities, A validation process may be performed on the constructed competitive risk model by performing cross-validation by dividing the entire subjects into fives, for the purpose of specificity(validity) validation. Hereinafter, a predictive power validation process of a metabolic syndrome risk prediction model will be described. Similarly, to the predictive power and validation process of the hypertension prediction model, the predictive power validation process of a metabolic syndrome risk prediction model may be performed using a total of three method. (1. cross validation with internal specificity(validity) using the ROC carve and the AUC value, 2. Comparison of an observed value and an expected value of hypertension for the calculated risk score value, and 3. Youden index, Distance to (0,1), and sensitivity and specificity(validity) equality for an optimal cut-point of hypertension risk)

Hereinafter, in order to verify the internal specificity(validity) of the metabolic syndrome risk model, an expected value of the metabolic syndrome was calculated and a number of cases of a total of 10 variables selected in the model was generated as matrix data (2¹⁰=1024).

An AUC value in the metabolic syndrome prediction model constructed using the 70% training set (3902 subjects) was calculated to be 0.7057 and the 95% confidence interval was 0.6932 to 0.7182. Further, the AUC value in the metabolic syndrome prediction model constructed using the 30% training set (22853 subjects) was calculated to be 0.6961 and the 95% confidence interval was 0.6765 to 0.7156.

FIG. 9N is a bar graph for comparing a metabolic syndrome risk score for 10 years estimated by the statistical probability model generating unit 130 and an incidence probability observed in actual study subjects according to the decile interval of the risk score.

(a) of FIG. 9O is a predictive power of a metabolic syndrome prediction model using repeatedly measured data of a training set (3902 subjects) and (b) is a predictive power of a metabolic syndrome prediction model using repeatedly measured data of a test set (2853 subjects).

The statistical probability model generating unit 130 may perform cross validation to verify the predictive power of the metabolic syndrome risk. According to the cross validation, similarly to the hypertension model and the overweight model, permutation was performed on the training set and the test set two times using a boot-strapping technique. Next, the cross validation was performed to identify whether the observed value and an expected value of the validation set match by applying the probability calculating method of the calculated model as it is. As a result as illustrated in the following drawing, as the validation value of the predictive power of the metabolic syndrome risk for the training set, the AUC is 0.7399 and the 95% confidence interval is 0.7394 to 0.7404. As the predictive power for the test set, the AUC was 0.6956 and the 95% confidence interval was 0.6949 to 0.6962.

(a) of FIG. 9P is a graph of a cross validation result of a predictive power of the metabolic syndrome risk using a boot-strap of a training set and (b) is a graph of a cross validation result of a predictive power of the metabolic syndrome risk using a boot strap of a test set.

The statistical probability model generating unit 130 confirms the optimal cut-point, sensitivity, and specificity(validity) using Youden index, Distance to (0,1), and the principle of sensitivity and specificity equality for the training set. As a method for calculating Youden index, a maximum value (sensitivity+specificity−1) was used and the maximum value at this time was 0.31692. According to this, it was continued that the cut-point was 0.29747, the sensitivity was 0.59065, and the specificity was 0.72869. According to the Distance to (0,1) method, the value was calculated by the following Equation. A minimum value calculated by the following Equation was 0.4453 and thus it was confirmed that the sensitivity was 0.61397 and the specificity was 0.70276. The sensitivity and specificity equality method means that the difference between the sensitivity and the specificity is minimum and the calculated minimum value was 0.00627 and thus the sensitivity was 0.64637 and the specificity was 0.65265.

Table 8 is an optimal cut-point a sensitivity, and a specificity(validity) of the metabolic syndrome using three methods.

TABLE 8

| | cut-point | Sensitivity | Specificity |
|---|---|---|---|
| Yoden index | 0.29747 | 0.59065 | 0.72869 |
| Distance to (0, 1) | 0.29391 | 0.61397 | 0.70276 |
| Sensitivity, Specificity | 0.28545 | 0.64637 | 0.65265 |
| equality | | | |

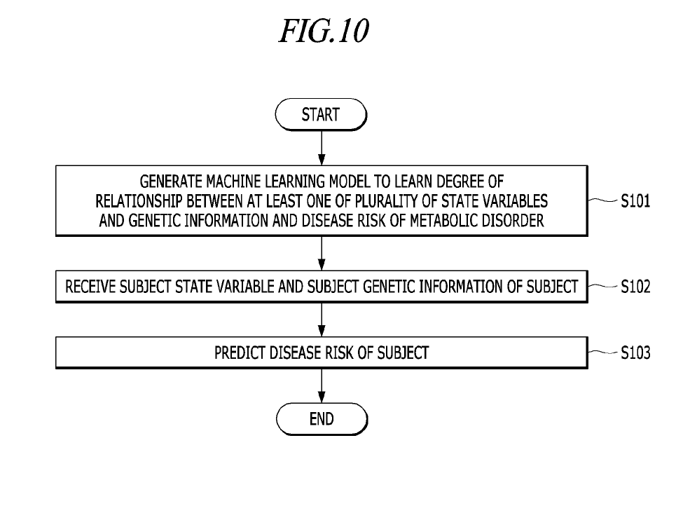

FIG. 10 is a schematic flowchart of a method for predicting a disease risk of metabolic disorders according to an exemplary embodiment of the present disclosure As the method for predicting a disease risk of metabolic disorders illustrated in FIG. 10. contents to be processed in respective units of the apparatus 100 for predicting a disease risk of metabolic disorders described with reference to FIGS. 1 to 9 will be schematically described. Therefore, even though the contents are not described below, the contents may be included in the description of the operation of the apparatus 100 for predicting a disease risk of metabolic disorders described with reference to FIGS. 1 to 9 or derived therefrom, so that detailed description thereof will be omitted.

Referring to FIG. 10, in step S101, the apparatus 100 for predicting a disease risk of metabolic disorders may generate a machine learning model which learns a degree of a relationship between at least one of a plurality of state variables and genetic information and a disease risk of metabolic disorders with a plurality of state variables including a living condition variable and a health condition variable of a patient with a metabolic disorder, generic information, and a disease risk of the metabolic disorders as inputs. Further, the apparatus 100 for predicting a disease risk of metabolic disorders may generate a statistical probability model probabilistically representing the disease risk of the metabolic disorders depending on whether there is at least one of the plurality of state variables and genetic information or the value, with the plurality of state variables, the genetic information, and the disease risk of the metabolic disorder of the patient with the metabolic disorder as inputs.

In step S102, the apparatus 100 for predicting a disease risk of metabolic disorders may receive a subject state variable and subject genetic information of the subject.

In step S103, the apparatus 100 for predicting a disease risk of metabolic disorders may predict a disease risk of the subject by applying a subject state variable and subject genetic information of the subject to the machine learning model.

The above-description of the present disclosure is illustrative only and it is understood by those skilled in the art that the present disclosure may be easily modified to another specific type without changing the technical spirit of an essential feature of the present disclosure. Thus, it is to be appreciated that the embodiments described above are intended to be illustrative in every sense, and not restrictive. For example, each component which is described as a singular form may be divided to be implemented and similarly, components which are described as a divided form may be combined to be implemented.

The scope of the present disclosure is represented by the claims to be described below rather than the detailed description, and it is to be interpreted that the meaning and scope of the claims and all the changes or modified forms derived from the equivalents thereof come within the scope of the present disclosure.

1. An apparatus for predicting a disease risk of metabolic disorder, the apparatus comprising:
a machine learning model generating unit which generates a machine learning model which learns a degree of a relationship between at least one of a plurality of state variables and genetic information and a disease risk of metabolic disorders with the plurality of state variables including a living condition variable and a health condition variable of a patient with a metabolic disorder, generic information, and the disease risk of the metabolic disorder as inputs;
an information input unit which receives a subject state variable and subject genetic information of the subject; and
a disease risk predicting unit which predicts a subject disease risk of the subject by applying the subject state variable and the subject genetic information of the subject to the machine learning model.

2. The apparatus for predicting a disease risk of metabolic disorder of claim 1, further comprising:
a statistical probability model generating unit which generates a statistical probability model probabilistically representing the disease risk of the metabolic disorders depending on whether there are at least one of the plurality of state variables and genetic information or a value, with the plurality of state variables, the genetic information, and the disease risk of the metabolic disorder of a patient with the metabolic disorder as inputs; and
a disease risk predicting unit which predicts a subject disease risk of the subject by applying the subject state variables and the subject genetic information to the machine learning model and the statistical probability model.

3. The apparatus for predicting a disease risk of metabolic disorder of claim 2, wherein the statistical probability model generating unit includes:
a basic statistical probability model generating unit which has the plurality of state variables, the genetic information, and a disease risk of the metabolic disorders of the patient with the metabolic disorder as inputs, selects at least one state variable associated with the metabolic disorder among the plurality of state variables, and generates a basic statistical probability model probabilistically representing the disease risk of the metabolic disorder for whether there is at least one state variable or the value; and
a weight statistical probability model generating unit which applies a weight to the disease risk of the metabolic disorder depending on whether there is genetic information associated with the metabolic disorder to generate a statistical probability model from the basic statistical probability model.

4. The apparatus for predicting a disease risk of metabolic disorder of claim 1, wherein when a first state variable among the plurality of state variables is assumed to be an input layer and a second state variable among the plurality of state variables is assumed to be a hidden layer, the machine learning model performs first learning to learn a degree of a relationship between the input layer and the hidden layer and when the hidden layer and the genetic information are assumed to be the input layer and the disease risk is assumed to be an output layer, performs second learning a degree of a relationship between the hidden layer and the output layer to learn a degree of a relationship between at least one of the plurality of state variables and genetic information and the disease risk of the metabolic disorder.

5. The apparatus for predicting a disease risk of metabolic disorder of claim 1, wherein when a previous state variable of the plurality of state variables is assumed to be an input layer and a current state variable of the plurality of state variables is assumed to be a hidden layer, the machine learning model performs first learning to learn a degree of a relationship between the input layer and the hidden layer and when the hidden layer and the genetic information are assumed to be the input layer and the disease risk is assumed to be an output layer, performs second learning a degree of a relationship between the hidden layer and the output layer to learn a degree of a relationship between at least one of the plurality of state variables and genetic information and the disease risk of the metabolic disorder.

6. The apparatus for predicting a disease risk of metabolic disorder of claim 1, wherein when a first state variable among the plurality of state variables and a previous hidden layer are assumed to be an input layer and a second state variable or a current state variable among the plurality of state variables is assumed to be a hidden layer, the machine learning model performs first learning to learn a degree of a relationship between the input layer and the hidden layer and when the hidden layer and the genetic information are assumed to be the input layer and the disease risk is assumed to be an output layer, performs second learning a degree of a relationship between the hidden layer and the output layer to learn a degree of a relationship between at least one of the plurality of state variables and genetic information and the disease risk of the metabolic disorder and
the first learning learns the degree of the relationship between the input layer and the hidden layer based on Equation 1 and
hₜ=tan h(Wₕₕhₜ₋₁+Wₓₕxₜ) [Equation 1]
in this case, hₜ is a hidden layer at a timing t, hₜ₋₁ is a hidden layer of a previous timing, xₜ is a first state variable, Wₕₕ is a first weight representing a degree of a first type of relationship between the input layer and the hidden layer, and Wₓₕ is a second weight representing a degree of a second type of relationship between the input layer and the hidden layer.

7. The apparatus for predicting a disease risk of metabolic disorder of claim 6, wherein the second learning learns a degree of a relationship between the hidden layer and the output layer, based on Equations 1 and 2, and
y=sigmoid(W_{yh}hₜ+W_{yz}z) [Equation 2]
in this case, y is the output layer, W_{yh} is a third weight representing a degree of the relationship between the hidden layer and the output layer, hₜ is a hidden layer, W_{yz} is a fourth weight representing a degree of the relationship between the genetic information of the input layer and the output layer, and z is the genetic information of the input layer.

8. The apparatus for predicting a disease risk of metabolic disorder of claim 1, wherein the machine learning model generating unit updates the weight to an error generated when the machine learning model to learn a degree of the relationship between at least one of the plurality of state variables and genetic information and the disease risk of the metabolic disorders is generated, based on equation 3, and
E=(t−y)²+λ∥W∥₂² [Equation 3]
E is a detected error of the machine learning model generating unit, t is whether the metabolic disorder occurs, y is a disease risk predicted through a machine learning model, and ∥W∥₂² is an L2 regular expression for preventing overfitting due to the error.

9. The apparatus for predicting a disease risk of metabolic disorder of claim 1, wherein the disease risk predicting unit visualizes a disease risk prediction result of the subject based on a predetermined classification category.

10. The apparatus for predicting a disease risk of metabolic disorder of claim 1, wherein the disease risk predicting unit provides disease preventive management information associated with a disease risk prediction result of the subject.

11. The apparatus for predicting a disease risk of metabolic disorder of claim 2, wherein when the metabolic disorder is hypertension, the statistical probability model generating unit generates a statistical probability model probabilistically representing a disease risk of hypertension according to values of the plurality of state variables including at least five of age, an education level, a monthly average income, anemia, proteinuria, glucose in urine, cholesterol, an amount of sodium intake, an amount of potassium intake, a drinking status, a smoking status, hyperlipidemia, fatty liver, allergic disease, arthritis, an uric acid level in blood, a family history of metabolic disorder, and whether to exercise.

12. The apparatus for predicting a disease risk of metabolic disorder of claim 2, wherein when the metabolic disorder is obesity, the statistical probability model generating unit generates a statistical probability model probabilistically representing a disease risk of the obesity according to values of the plurality of state variables including at least five of age, an education level, a past history of hyperlipidemia, a past history of myocardial infarction, a past history of fatty liver, a past history of cholecystitis, a past history of allergy, a thyroid gland disease, arthritis a blood pressure, whether to exercise, an amount of sodium intake compared with an amount of energy intake, an amount of protein intake, an amount of fat intake, proteinuria, a total cholesterol, a fasting blood sugar, a drinking status, a smoking status, an uric acid level in blood, and a family history of metabolic disorder.

13. The apparatus for predicting a disease risk of metabolic disorder of claim 2, wherein when the metabolic disorder is diabetes, the statistical probability model generating unit generates a statistical probability model probabilistically representing a disease risk of the diabetes according to values of the plurality of state variables including at least five of an education level a marriage state, an occupation, an income, a gender, an age a past history of hypertension, a past history of hyperlipidemia a past history of myocardial infarction, a past history of chronic gastritis, a past history of fatty liver, a past history of cholecystitis, a past history of chronic bronchitis, a past history of asthma, a past history of allergy, arthritis, a past history of osteoporosis, a past history of cataract, a past history of depressive disorder, a past history of thyroid gland disease, a number of exposure to passive smoking, total alcohol intakes, a number of exercises, an age of first birth, a past history of gestational diabetes, a past history of reduced abortion, a past history of birth of fetal macrosomia, whether to take oral contraceptive pill, a family history of diabetes, a family history of angina pectoris, a family history of stroke, a current subjective health condition, a quality of sleep, hematuria, fat, carbohydrate, vitamin, zinc, a weight, a waist size, a hip circumference, a pulse rate, a diastolic blood pressure, a systolic blood pressure, and a body mass index.

14. The apparatus for predicting a disease risk of metabolic disorder of claim 2, wherein when the metabolic disorder is a metabolic syndrome, the statistical probability model generating unit generates a statistical probability model probabilistically representing a disease risk of metabolic syndrome according to values of the plurality of state variables including at least five of an age, a gender, an education level, a monthly average income, ALT, anemia, proteinuria, sodium intake, potassium intake, energy intake, whether to exercise, a pack year of smoking, a past history of myocardial infarction, a past history of fatty liver, a past history of cholecystitis, an allergic disease, a past history of thyroid gland disease, arthritis, an uric acid level in blood, and a family history of metabolic disorder.

15. A method for predicting a disease risk of metabolic disorder, the method comprising:
generating a machine learning model which learns a degree of a relationship between at least one of a plurality of state variables and genetic information and a disease risk of metabolic disorders with the plurality of state variables including a living condition variable and a health condition variable of a patient with a metabolic disorder, generic information, and the disease risk of the metabolic disorders as inputs;
receiving a subject state variable and subject genetic information of the subject; and
predicting a disease risk of the subject by applying the subject state variable and the subject genetic information of the subject to the machine learning model.
